(19) United States
(12) Patent Application Publication
(10) Pub. No.: US 2003/0065296 A1
(43) Pub. Date: Apr. 3, 2003

(54) Absorbent material of water absorbent polymer, thermoplastic polymer, and water and method for making same

(76) Inventors: Thomas A. Kaiser, Greensboro, NC (US); Frank R. Parchen JR., Chadds Ford, PA (US); Thomas E. Hopkins, Greenville, SC (US)

Correspondence Address: JENKINS & WILSON, PA, 3100 TOWER BLVD, SUITE 1400, DURHAM, NC, 27707, US

(21) Appl. No.: 09/793,005

(22) Filed: Feb. 26, 2001

(51) Int. Cl. A61F 13/15; A61F 13/20; B32B 5/16; B32B 9/00; B32B 15/02; B32B 17/02; B32B 19/00; B32B 21/02; B32B 23/02; B32B 27/02

(52) U.S. Cl. 604/368; 604/370; 604/372; 428/402

(57) ABSTRACT

An absorbent material is disclosed, containing at least about 30 weight percent of a superabsorbent polymer, a thermoplastic polymer binder resin, and about 0.1 to about 10 weight percent water. The absorbent material absorbs deionized water to at least about 70 percent of maximum capacity within about 20 minutes after exposure to the deionized water. The method of making the absorbent material includes combining binder resin and absorbent polymer in a twin screw extrusion mechanism, compounding and driving the composition toward exit openings, extruding the composition through the exit openings, and preferably rapidly cooling the extrudate with non-liquid quenching means. The quenched or non-quenched extrudate may be made in the form of a pellet, film, or fibrous strand.

### TECHNICAL FIELD

The present invention relates, in general, to an absorbent material, and to a method for making same. More particularly, the present invention relates to absorbent materials of superabsorbent polymers, namely polymers that absorb at least 20 times their weight in water, and thermoplastic polymers, especially absorbent materials in the form of films, which absorbent materials have superior water-absorbency properties and superior water-blocking properties as compared to previously known absorbent materials.

Definitions of Abbreviations

| Abbreviation | Definition |
|---|---|
| Abs | Absorption |
| ASTM | American Society for Testing |
| | Materials |
| Avg | Average |
| C | centigrade |
| X-linking | cross-linking |
| cc | cubic centimeter |
| DI | deionized |
| DW | demand wetability |
| EAA | ethylene alkyl acrylate |
| | copolymer |
| EBA | ethylene butyl acrylate |
| | copolymer |
| EEA | ethylene ethyl acrylate |
| | copolymer |
| EVA | ethylene vinyl acetate |
| | copolymer |
| g | gram |
| HDPE | high density polyethylene |
| HMI | high melt index |
| LLDPE | linear low density polyethylene |
| LDPE | low density polyethylene |
| LMI | low melt index |
| MDPE | medium density polyethylene |
| μm | micrometer |
| mm | millimeter |
| min | minute |
| % | percent |
| PP | polypropylene |
| SAP | superabsorbent polymer, a |
| | polymer that absorbs at least |
| | 20 times its weight in water |
| TB | tea bag |
| TPBR | thermoplastic polymer binder |
| | resin |
| VLDPE | very low density linear low |
| | density polyethylene |
| wt | weight |

### BACKGROUND ART

SAPs, namely highly water-swellable polymers, typically are prepared from an aqueous mixture of monomers. Usually, one or more network X-linking agents are incorporated into the monomer mixture. When the polymerization has ended, the viscous resultant is dried and subjected to mechanical grinding to create a desired particle size distribution for the particular SAP.

SAPs are made by two methods, namely the solvent or solution polymerization method and the inverse suspension or emulsion polymerization method. In the solvent or solution polymerization method, an aqueous solution, for instance of partially neutralized monomer of acrylic acid, methacrylic acid, or maleic acid and a multi-functional network X-linking agent, is converted to a gel by radical polymerization. On the other hand, in the inverse suspension or emulsion polymerization method, an aqueous solution, for instance of partially neutralized monomer of acrylic acid, methacrylic acid, or maleic acid, is dispersed in a hydrophobic organic solvent by employing colloids or emulsifiers, and polymerization is started by radical initiators. Network X-linking may be accomplished by dissolving a polyfunctional X-linking agent in the monomer solution. The resultant from either method is dried, ground, and screened to the desired particulate size.

As discussed in more detail below, SAPs are useful in various absorbent articles, due to the ability of the SAPs to absorb liquids such as water and bodily liquids (i.e., urine, blood, menstrual flow) in a ready manner.

For example, absorbent polymers have been employed in sanitary articles (such as throwaway baby diapers, incontinence garments, bed pads, sanitary napkins, bandages, wound dressings, surgical drapes, or clean-up pads) and other applications requiring absorption of a large amount of liquids.

Such other applications of absorbent polymers include a sealing composite (such as in wine corks, boats, houses, plumbing, water stops, caulking, gaskets, hydraulic cement, gutters, flat tire repair, or water proofing composites, for instance, a water proofing composite between concrete blocks that make up the wall of underwater tunnels, like the Channel Tunnel connecting England and France), a tape (forwater blocking in fiber optic cables or power transmission cables), an agricultural article (such as a controlled release carrier for insecticides, herbicides, and/or pesticides, as shown in Levy U.S. Pat. Nos., 4,818,534, 4,983,389, 4,983,390, 4,985,251, 5,567,430, and 5,824,328, all assigned to Stockhausen GmbH and Co. KG, or such as STOCKOSORB®, which is a SAP marketed by Stockhausen for use as a soil amendment in agricultural fields to improve the capability of soils to keep water and nutrients near or with the roots of plants), a filtration sheet (such as for removal of water or moisture from gasoline, fuel, oil, organic solvent, and the like), an absorbent liner (such as in food packaging, and depending on the particular polymer in accordance with the regulations of the U.S. Food and Drug Administration, the liner may or may not be able to be in direct contact with the food, or such as in astroturf, dikes, erosion control, irrigation systems, book repair, hydraulic devices, roofs, gas tanks, slaughter houses, mildew protectors, or shipping containers), a packaging material article for packaging items requiring moisture (such as cut flowers), fillers (such as in recording materials, batteries, brush fibers, fire place logs, furniture, gel transformers, tractor tires, diet pills, cast reinforcement, ballast, capillary devices, humidity control devices, or indoor/outdoor carpets), and coatings (such as antifouling coatings, paint additives, or thickeners).

More specifically, any damage from moisture can be localized and minimized with application of such absorbent polymers for swelling/water blocking in cables, so that undesirable moisture that penetrated the outer casing of the cable can be absorbed by the absorbent polymer and blocked from further travel in the cable, as by swelling of the absorbent polymer. Moisture within a cable can disrupt transmission through the cable and is particularly undesirable in colder climates because freezing of the moisture that creeps into the cable could result in the deflection or termination of the signal carried by the cable. Moisture is particularly a problem with respect to transmissions by fiber optic cables.

Various techniques have been used to prevent moisture from entering a cable and to minimize the migration of such moisture if it does penetrate into the cable. One method to minimize water migration in cables is to impregnate an already made tape with a powdered superabsorbent polymer, and then either wrap the impregnated tape around a core or place it longitudinally within the cable assembly, such as shown in Arroyo U.S. Pat. No. 4,867,526; Arroyo et al. U.S. Pat. No. 4,909,592; Arroyo et al. U.S. Pat. No. 5,133,034; Arroyo et al. U.S. Pat. No. 5,321,788; Arroyo U.S. Pat. No. 5,410,629; Yuchioke et al. U.S. Pat. No. 4,703,998; and Pasta et al. U.S. Pat. No. 5,261,021. Similarly, cable coatings can be doped with powdered superabsorbent polymers, as shown in Elion U.S. Pat. No. 4,525,026.

Another method to minimize the migration of penetrating moisture in cables is to coat a yarn or other strengthening member with an absorbent polymer and then place the coated yarn in or around a cable bundle to absorb any liquid in the bundle. Such uses are described in Arroyo U.S. Pat. No. 4,913,517; Guersen et al. U.S. Pat. No.5,342,686; Arroyo et al. U.S. Pat. No. 5,389,442; and Guersen et al. U.S. Pat. No. 5,264,251.

It is desirable to have an absorbent material of an absorbent polymer that can be directly used within the cable without having to impregnate or to coat another already made structure with the absorbent polymer in order to enable the absorbent polymer to be placed in the cable in order to effect a sufficient concentration of absorbent polymer to absorb moisture efficiently and to minimize its migration through the cable.

It has been generally thought desirable to combine an absorbent polymer with a thermoplastic polymer binder resin to create such an absorbent material that can be formed into a film, sheet, or the like for use within a cable, as well as for use in other applications.

However, it is against conventional wisdom to form a film by incorporation of an absorbent polymer, which is hydrophilic, into a thermoplastic polymer binder resin, which is hydrophobic, while retaining not only the absorbency of the absorbent polymer but also the moldability and the self-supporting form retention properties of the thermoplastic polymer binder resin. Past attempts to incorporate an absorbent polymer into thermoplastic polymer binder resins, and then extrude, cast, or mold a sheet or a film from the mixture, have been without success because of problems blending with the substances and problems with attaining sufficient concentrations of absorbent polymer to be effective. Much of the absorbency of the absorbent polymer is often lost when it is incorporated into such a thermoplastic polymer binder resin. Nevertheless, the more of absorbent polymer that can be placed within a cable, then the more efficient is the moisture absorption and minimization of the migration of the moisture through the cable.

More specifically, an attempt to create an absorbent composition that can be directly used in cables and other applications includes the incorporation of a block copolymer of styrene/rubber together with a superabsorbent polymer to form an absorbent material which will retain appropriate viscosity, as is illustrated in Connole et al. U.S. Pat. No. 5,306,867, assigned to AT&T Bell Laboratories. More specifically, this patent discloses a gel of 10 parts by weight superabsorbent polymer mixed with oil and polyethylene in a styrene/rubber block copolymer (such as styrene-ethylene-butylene/rubber diblock), and used to fill in the voids within a bundle of cables.

Also, the composition of Doi et al. U.S. Pat. No. 4,977,211, assigned to Mitsubishi Petrochemical Co., appears to be able to incorporate large concentrations of superabsorbent polymer, but it is noted that suitable absorbent and moldability properties of this composition when molded into a sheet are attained only with significant presence of a copolymer rubber (such as an ethylene-propylene rubber copolymer). Significantly, Doi et al. '211 teaches that use of a superabsorbent polymer with a polyolefin resin is a disadvantage because of a low water absorption ratio and that the inclusion of a copolymer rubber is required to allow effective concentrations of the superabsorbent polymer to be present in the molded sheet to provide sufficient absorbency properties.

Another example of such an attempt at incorporation is shown in Ehrhardt et al. U.S. Pat. No. 5,419,955, assigned to Hoechst Celanese Corporation, where absorbent material is fabricated by forming a liquid mixture of a polyester resin matrix and a plasticizer. A powdered SAP is introduced into the liquid plasticizer/matrix mixture, which is then extruded or cast into a sheet. This may result in some immobilization of SAP particles within a matrix, but only at the expense of using a plasticizer, which can be expensive and may pose significant health and environmental concerns. Moreover, the 18% by weight plasticizer illustrated, which is a very high %, can cause slippage in an extruder when large amounts of resin are employed on a factory scale as opposed to the small amounts used on a laboratory scale.

A significant factor in the use of absorbent polymers to prevent water migration is the rate at which the absorbent polymer can absorb water. In other words, the presence of an absorbent material within a cable has a greater effectiveness at stopping the migration of water if the absorbent material has a very fast rate of absorbency to enable quick blockage of the water movement.

SAPs have been developed with improved absorbency characteristics, such as faster rate of water absorption and higher quantity of water absorbed. Typically, these SAPs are generally provided as cross-linked polymers in particle form with no thermoplasticity. This typical lack of thermoplasticity makes it difficult to mold, to cast, or to extrude the SAP particles into a film, sheet, or the like.

Generally, SAP particles have diameters between 100 μm and 850 μm, the standard size for these particles. When SAPs are created and ground into particles for use, those large particles with diameters greater than about 850 μm (referred to as “overs”) are generally recycled. Those small particles with diameters less than about 100 μm (referred to as “fines”) are generally unsuitable for most SAP applications. Such small particles have a greater tendency to become airborne as dust particles, and thus, are difficult to handle and to use in superabsorbent applications. Also, the superabsorbent fines generally reach absorbent capacity more quickly. This can lead to a gel blocking effect, in which the outward particles absorb to capacity very quickly and prevent liquid penetration to inward particles. Thus, gel blocking can prevent the use of fines in certain applications.

Therefore, it is desirable to provide an absorbent material with improved formability that may be directly molded, cast, or extruded into a film, sheet, or the like, with minimum loss of integrity of the material, with a high concentration of superabsorbent polymer therein, with a high absorbent capacity, with a high speed of moisture absorbency, and with a freedom from exhibiting significant gel blocking. It is also desirable to create such an absorbent material using superabsorbent polymer “fines”.

### SUMMARY AND OBJECTS OF THE INVENTION

The present invention provides an absorbent material with at least about 30 wt % of a superabsorbent polymer. The superabsorbent polymer preferably includes at least one acrylic acid derivative monomer. The absorbent material further has a thermoplastic polymer binder resin, preferably a polyolefin resin, and also has about 0.1 to about 10 wt % water. Preferably, an inorganic particulate useful as a flow agent, such as fumed silica, may also be present in a minor amount. Typically, this absorbent material absorbs liquid, i.e., deionized water, to at least about 70% of its capacity within about 20 minutes after exposure to the liquid.

Also, the present invention includes a composition of superabsorbent polymer, thermoplastic polymer binder resin, and water for making such an absorbent material. The particle size of superabsorbent polymer employed may range up to about 1000 μm, and may be preferably less than about 100 μm.

Moreover, the present invention provides an article of manufacture comprising an absorbent material, having at least about 30 weight percent of superabsorbent polymer, thermoplastic polymer binder resin, and about 0.1 to about 10 weight percent water, wherein the absorbent material absorbs deionized water to at least about 70 percent of capacity within about 20 minutes after exposure to the deionized water. Preferably, the article may be selected from the group consisting of a sanitary article, a sealing composite article, a water blocking tape article, an agricultural article, a filtration sheet article, an absorbent liner article, a packaging material article for packaging items requiring moisture, a filler material article, and a coating material article.

Additionally, the present invention provides a method of making an absorbent material from the composition noted in the above paragraph, wherein the thermoplastic polymer binder resin, the superabsorbent polymer, and the water are combined, for instance, in a twin screw extrusion mechanism. The composition is driven toward openings through which it is forced. The warm extrudate may then be rapidly cooled with non-liquid quenching means (particularly with non-aqueous quenching means), such as cold air, a chilled roller, a cold stainless steel belt, and the like, and formed into, for example, pellets, cast film, or blown film, or not quenched and formed into, for example, melt-blown film, fiber strands, or another fixed shape, such as by injection molding. The melt blown fiber process to make fiber strands is well known,just as processes to make film are well known. Preferably, the resultant absorbent material is formed into an article selected from the group consisting of a sanitary article, a sealing composite article, a water blocking tape article, an agricultural article, a filtration sheet article, an absorbent liner article, a packaging material article for packaging items requiring moisture, a filler material article, and a coating material article.

The invention further provides a method of providing for the prevention of the migration of liquid within a cable such as the type having multiple components.

Thus, it is an object of the present invention to provide an absorbent material with improved formability that may be directly molded, cast, or extruded into a film, sheet, strand, or the like, with minimum loss of integrity of the absorbent material, with a high concentration of superabsorbent polymer therein, with a high absorbent capacity, and with a high speed of moisture absorbency.

It is an advantage of the present invention to create such an absorbent material that in one embodiment (a) uses superabsorbent polymer “fines” that are otherwise difficult to contain and (b) forms the superabsorbent fines appropriately, such as into a sheet, film, fibers, pellets, etc.

Some of the objects of the invention having been stated, other objects and advantages will become evident as the description proceeds, when taken in connection with the Drawings and the Laboratory Examples described below.

### BRIEF DESCRIPTION OF THE DRAWINGS

FIG. 1 is a representation of a twin screw extrusion mechanism utilized in practicing the preferred embodiment of the method of the present invention;

FIG. 2 is a representation of the internal structure of a typical single core cable embodying a preferred embodiment of the present invention; and

FIG. 3 is a cross-section of a typical multiple core cable embodying a preferred embodiment of the present invention.

### DETAILED DESCRIPTION OF THE INVENTION

The present invention includes an absorbent material, a method of making the absorbent material, and an article of manufacture incorporating the absorbent material.

The absorbent material of the present invention contains at least about 30 wt % of a SAP, which may be any SAP, but preferably is a SAP that includes at least one monomer selected from acrylic acids. More preferably, the SAP includes at least one monomer selected from salts of acrylic acids, and most preferably the SAP is sodium polyacrylate.

The SAPs useful in the absorbent material of the present invention may be manufactured on a large scale by continuous or discontinuous processes of the prior art for making SAPs. For instance, the SAP may be made by the solvent or solution polymerization technique or may be made by the inverse suspension or emulsion polymerization technique, which are well known techniques as discussed above.

Both techniques typically begin with an aqueous monomer solution, for instance of acrylic acid monomer, which is neutralized at some point. With solvent polymerization, the acid solution also contains a multi-functional network X-linking agent and is converted into a gel by radical polymerization. The gel is then dried. In contrast, with inverse suspension or emulsion polymerization, the acid solution is dispersed in a hydrophobic organic solvent by employing colloids or emulsifiers. Next, polymerization is initiated with radical initiators. After completion of polymerization, water is azeotropically removed from the reaction mixture, and the product is then filtered and dried. Network X-linking typically is accomplished by dissolving a poly-functional network X-linking agent in the monomer solution.

Thus, the SAP may be obtained by polymerizing at least about 10%, more preferably about 25%, and even more preferably about 55 to about 99.9% by weight of monomers having olefinically-unsaturated groups, such as acrylonitrile groups, anhydride groups, carboxylic acid groups, or sulfonic acid groups. Such carboxylic acid groups include, but are not limited to, acrylic acids and methacrylic acids. An example of a sulfonic acid group is 2-acrylamido-2-methylpropane sulfonic acid. The groups are typically present as salts, such as sodium, potassium, or ammonium salts, i.e., the sodium acrylate salt of acrylic acid.

The acid groups are typically neutralized to at least about 25 mol %. Preferably, the extent of neutralization is to at least about 50 mol %. More particularly, the preferred SAP has been formed from X-linked acrylic acid or methacrylic acid, which has been neutralized to an extent of about 50 to about 80 mol %. Suitable neutralizing agents are hydroxides and/or carbonates of alkaline earth metals and/or alkali metals, for instance, of Na, K, Li, Be, Mg, Fe, Co, Ni, and the like. Thus, the preferred SAP is sodium polyacrylate.

Additional useful monomers for making the SAPs include ethers, imides, amides (such as acrylamide, methacrylamide, and dimethyl aminopropyl acrylamide), maleic acid, maleic anhydride, vinyl chloride, vinyl alcohol, styrene, esters (such as hydroxyethyl acrylate, hydroxypropyl acrylate, hydroxypropyl methacrylate, and dimethyl-aminoalkyl-methacrylate), and acrylamidopropyl trimethylammonium chloride.

Suitable network X-linking agents useful in making the SAPs are those which have at least two ethylenically unsaturated double bonds, those which have one ethylenically unsaturated double bond and one functional group reactive toward acid groups, and those which are multi-functional, i.e., have several functional groups reactive toward acid groups. Suitable kinds of network X-linking agents include, but are not limited to, acrylate, and methacrylate of polyols (such as butanediol diacrylate, hexanediol dimethacrylate, polyglycol diacrylate, trimethylpropane triacrylate, allyloxy polyethylene glycol diacrylate, and ethoxylated trimethylolpropane triacrylate), allyl acrylate, diallyl acrylamide, triallyl amine, diallyl ether, methylenebisacrylamide, glycerol dimethacrylate, N-methylol methacrylamide, and N-methylolacrylamide. Suitable kinds of network X-linking agents that are multi-functional include, but are not limited to, alcohols, amines, and epoxides, such as glycol, propylene glycol, glycerol, ethylene diamine, hexamethylene diamine, glycerol polyglycidal ether, and resorcinol diglycidal ether. These network X-linking agents are distinguished from and not to be confused with the surface X-linking agents discussed below.

Furthermore, depending on the desired end use, the SAP may have a water-soluble polymeric component. The content may range from above 0 up to about 30% by weight of a component that includes, but is not limited to, partially or complete saponified polyvinyl alcohol, polyvinyl pyrrolidone, starch, starch derivatives, polyglycols, polyacrylic acids, and combinations thereof. The molecular weight of the component is not critical, provided that it is water-soluble. Preferred water-soluble polymeric components are starch, polyvinyl alcohol, and mixtures thereof. Preferably, the content of the water-soluble polymeric component in the SAP ranges from about 1 to about 5% by weight, especially if starch and/or polyvinyl alcohol are present as the water-soluble polymeric component. Also, the water-soluble polymeric component may be present as a graft polymer having the acid-groups-containing polymer.

It is known from Dahmen U.S. Pat. No. 5,409,771, assigned to Chemische Fabrik Stockhausen GmbH, to coat SAP particles with an alkylene carbonate followed by heating to effect surface X-linking. The SAPs useful in the present invention may be surface X-linked.

More specifically, as described in U.S. Pat. No. 5,409,771, in order to coat the SAP particles with a surface X-linking agent (such as an alkylene carbonate, a diol, a diamine, or a diepoxide), the SAP particles may be mixed with an aqueous-alcoholic solution of the surface X-linking agent. The amount of alcohol is determined by the solubility of the surface X-linking agent and is kept as low as possible for technical reasons, for instance, protection against explosions. Suitable alcohols are methanol, ethanol, butanol, or butyl glycol, as well as mixtures of these alcohols. The preferred solvent is water which typically is used in an amount of 0.3 to 5.0% by weight, relative to the particulate SAP. In some instances, the surface X-linking agent is dissolved in water, without any alcohol. It is also possible to apply the surface X-linking agent from a powder mixture, for example, with an inorganic carrier material, such as SiO₂, (see, lines 51-54 of column 4 of U.S. Pat. No. 5,409,771).

The following are suitable as surface X-linking agents. Alkylene carbonates are preferred and the following may be used as alkylene carbonates, e.g., 1,3-dioxolan-2-one, 4-methyl-1,3-dioxolan-2-one, 4,5-dimethyl-1,3-dioxolan-2-one, 4,4-dimethyl-1,3-dioxolan-2-one, 4-ethyl-1,3-dioxolan-2-one, 4-hydroxyethyl-1 ,3-dioxolan-2-one, 1,3-dioxan-2-one, 4-methyl-1,3-dioxan-2-one, 4,6-dimethyl-1,3-dioxan-2-one, 1,3-dioxepan-2-one, and combinations thereof. A diol is 1 ,4-butanediol diglycidyl ether. A diamine is 1,5-diaminopentane. A diepoxide is 1,3-butadiene diepoxide.

To achieve the desired surface X-linking properties, the surface X-linking agent should be distributed evenly on the SAP. For this purpose, mixing is effected in suitable mixers, such as fluidized bed mixers, paddle mixers, milling rolls, or twin-worm-mixers, or on a small scale, a standard household-type KITCHEN AID® mixer may be used.

Further according to U.S. Pat. No. 5,409,771, the thermal treatment which follows the coating treatment is generally at a temperature between 150 and 300° C. However, if the preferred alkylene carbonates are used, then the thermal treatment is at a temperature between 180 and 250° C. The treatment temperature depends on the dwell time and the kind of surface X-linking agent, such as an alkylene carbonate. At a temperature of 150° C., the thermal treatment typically is carried out for an hour or so. On the other hand, at a temperature of 250° C., a few minutes, e.g., 0.5 to 5 minutes, typically are sufficient. The thermal treatment may be carried out in conventional dryers or ovens, such as rotary kilns, fluidized bed dryers, disk dryers, or infrared dryers.

The absorbent material of the present invention further includes a thermoplastic polymer binder resin that enables the absorbent material to be shaped and thus substantially to retain such shape. Thus, the absorbent material can be ready formed into a self-supporting film, as described in detail below. Self-supportability is a feature even in the absence of a polyester and/or a rubbery polymer (i.e., styrene-ethylene-butylene rubber block copolymer or ethylene-propylene rubber copolymer) which two polymers are known for rigidity.

More particularly, the thermoplastic polymer binder resin useful in the absorbent material of the present invention preferably is a polymer that has at least one hydrophobic monomer, and more preferably is a polyolefin.

The term “polyolefin” as used herein generally includes, but is not limited to, materials such as polyethylene, ethylene vinyl acetate copolymer and the like, the homopolymers, copolymers, terpolymers, etc., thereof, and blends and modifications thereof. The term “polyolefin” shall include all possible structures thereof, which includes, but is not limited to, isotatic, synodiotactic and random symmetries.

The term “polyethylene” as used herein, which “polyethylene” is a type of polyolefin that may be employed in the absorbent material of the present invention, refers to families of resin obtained by substantially polymerizing the gas ethylene, C₂H₄. By varying the comonomers, catalysts and methods of polymerization, properties such as density, melt index, crystallinity, degree of branching, molecular weight, and molecular weight distribution can be regulated over wide ranges. Further modifications are obtained by other processes, such as halogenation, and compounding additives. Low molecular weight polymers of ethylene are fluids used as lubricants; medium weight polymers are generally miscible with paraffin; and the high molecular weight polymers are resins generally used in the plastics industry. Polyethylenes having densities ranging from about 0.900 g/cc to about 0.935 g/cc are called low density polyethylenes (LDPE) while those having densities from about 0.935 g/cc to about 0.940 g/cc are called medium density polyethylenes (MDPE), and those having densities from about 0.941 g/cc to about 0.965 g/cc and over are called high density polyethylenes (HDPE). The older, classic low density types of polyethylenes are usually polymerized at high pressures and temperatures whereas the older, classic high density types are usually polymerized at relatively low temperatures and pressures.

The term “linear low density polyethylene” (LLDPE) as used herein, for a type of polyethylene which may be employed in the absorbent material of the present invention, refers to the newer copolymers of a major amount of ethylene with a minor amount of one or more comonomers selected from C₃ to about C₁₀ or higher alpha olefins such as butene-1, 4-methyl pentene-1, hexene-1, octene-1, etc. in which the molecules thereof comprise long chains with few side chains or branched structures achieved by low pressure polymerization. The side branching which is present will be short as compared to non-linear polyethylenes. The molecular chains of a linear polymer may be intertwined, but the forces tending to hold the molecules together are physical rather than chemical and this may be weakened by energy applied in the form of heat. LLDPE polyethylene has a density preferably in the range from about 0.911 g/cc to about 0.935 g/cc, more preferably in the range of from about 0.912 g/cc to about 0.928 g/cc for film making purposes. The melt flow index of LLDPE generally ranges from between about 0.1 to about 15 grams per 10 minutes and preferably between from about 0.5 to about 3.0 grams per 10 minutes. LLDPE resins of this type are commercially available and are manufactured in low pressure vapor phase and liquid phase processes using transition metal catalysts. LLDPE is well known for its structural strength and anti-stresscracking properties. Also, LLDPE is known for its favored properties in the heat shrink process, and this is well suited to make a heat shrinkable film as discussed below. Also, very low density linear low density polyethylenes (VLDPE) may be employed, and such have a density from about 0.910 g/cc to about 0.860 g/cc, or even lower.

The term “ethylene alkyl acrylate copolymer” (EAA) as used herein for a type of polyolefin refers to a copolymer formed from ethylene and alkyl acrylate wherein the ethylene derived units in the copolymer are present in major amounts by weight and the alkyl acrylate derived units in the copolymer are present in minor amounts by weight. Thus, the term “ethylene methyl acrylate copolymer” (EMA) as used herein for a type of polyolefin, refers to a copolymer formed from ethylene and methylacrylate monomers. The term “ethylene ethylacryalte copolymer” (EEA) as used herein for a type of polyolefin, refers to a copolymer formed from ethylene and ethyl acrylate monomers. The term “ethylene butyl acrylate copolymer” (EBA) as used herein for a type of polyolefin, refers to a copolymer formed from ethylene and butyl acrylate monomers. Many suitable EBAs are commercially available and these have a butyl acrylate content from about 3% to about 18% by weight.

The term “polypropylene” (PP) as used herein for a type of polyolefin refers to polymers of propylene and includes homopolymers, copolymers, such as for example block, graft, random, and alternating copolymers, terpolymers, etc., and blends and modifications thereof.

The term “ethylene vinyl acetate copolymer” (EVA) as used herein for a type of polyolefin refers to a copolymer formed from ethylene and vinyl acetate monomers wherein the ethylene derived units in the copolymer are present in major amounts by weight and the vinyl acetate (VA) derived units in the copolymer are present in minor amounts by weight. EVA is also known for having structural strength, as LLDPE does. For film forming purposes, it is desirable that the VA content of the EVA be from about 4% to about 30% by weight, as when an EVA has a higher VA content the EVA behaves more like a glue or adhesive.

Preferably, the polyolefin is an EVA. It is noted that acceptable results have been observed when EVA having a low melt index has been used and better results have been observed when EVA having a high melt index has been used. The melt index of the EVA should be above about 300, more preferably above about 400.

Nevertheless, blends of all families of polyolefins, such as blends of PP, EVA, EMA, EEA, EBA, HDPE, MDPE, LDPE, LLDPE, and/or VLDPE, may also be advantageously employed.

The SAP is substantially homogenously blended together with the thermoplastic polymer binder resin such that pocketing of the SAP is essentially eliminated. This maintains the integrity and continuity of the absorbent material. Preferably, blending is accomplished with a twin screw extruder.

Pocketing in this sense refers to the agglomeration of SAP into discrete regions within the absorbent material. Pocketing of the SAP may lead to gel blocking or structural weaknesses in those areas in which pocketing occurs. Such structural weaknesses could lead to failure of that region when the blend of SAP and thermoplastic polymer binder resin is extruded into a film or formed into another shape.

SAP concentrations of up to about 60 wt % have been attained with the present invention, whereas known absorbent materials have been limited, in general, to less than 40 wt % SAP. It is anticipated that higher concentrations, up to at least about 75%, and even about 90 wt %, are possible with the present invention. When the concentration of SAP exceeds about 50 wt %, marked improvement in performance is noted. The absorbent material tends to absorb liquid at a rate much closer to that seen with pure SAP.

Without in any way meaning to be limited to a particular explanation for this phenomenon, it is noted that at concentrations of SAP less than about 50 wt %, the SAP makes up “islands” in a “sea” of thermoplastic polymer binder resin which physically can prevent the SAP from expanding fully, which limits its absorbency rate. On the other hand, at concentrations of SAP greater than about 50 wt %, the thermoplastic polymer binder resin is now the “island” in a “sea” of SAP, and thus, the rate of absorption is not hindered as much as when the concentration of SAP is less than about 50 wt %. In other words, for concentrations of SAP under about 50 wt %, the absorbency rate will be a lesser % than the absorbency rate for the same amount of SAP alone than would be the case for concentrations of SAP over about 50 wt %. This possibly explains the unexpected increase in absorbency rate with higher SAP concentrations while still being blended with the thermoplastic polymer binder resin in the absorbent material.

Nevertheless, absorbent materials with SAP concentrations of as little as about 30 wt % can still exhibit satisfactory characteristics, depending on the end use. Thus, the absorbent material is made from a composition having at least about 30 wt % of SAP particles.

The SAP particles may be of any of the typical sizes for SAP particle, which can range up to about 1000 μm or more, and thus, may be overs, which are at least about 850 μm, or may be large particles that are not quite overs, i.e., at least about 800 μm. Typically, the SAP particles are of standard particle size ranging from about 300 μm to about 500 μm in diameter, and are preferably fines that are less than about 200 μm in diameter, and more preferably less than about 100 μm in diameter. Thus, the present invention uses, in one embodiment, SAP particles which are fines. Suitable SAP fines are Na polyacrylate fines that are marketed by Stockhausen under the trade name FAVOR 800 SF, which has a mean particle size ≦100 μm. Also, FAVOR 800 HS may be employed, which contains a minor amount of the inorganic particulate, silica, but is otherwise like FAVOR 800 SF.

As discussed above, the fines in their pure state may more easily become airborne, as dust, than larger sized particles. Hence, the fines are more difficult with which to work. Further, the fines generally absorb liquids at a faster rate and are subject to gel blocking. Gel blocking occurs when the outward fines absorb to capacity very quickly and prevent fluid from penetrating to the inward fines. This reduces the absorbency of effectiveness of the fines. By immobilizing the fines in the homogenous blend with the thermoplastic polymer binder resin in the absorbent material, the fines can be effectively utilized as a SAP and are much less susceptible to the gel blocking effect.

The use of the smaller sized particles in an embodiment of the present invention also contributes to the homogenous blending of the SAP particles with the thermoplastic polymer binder resin. Preferably, the SAP particles have a minor amount of fumed silica dispersed with them in order to minimize agglomeration of the SAP particles during the homogenous blending of the SAP particles and the thermoplastic polymer binder resin.

The absorbent material further includes about 0.1 to about 10 wt % water, preferably about 0.5 to about 5 wt % water.

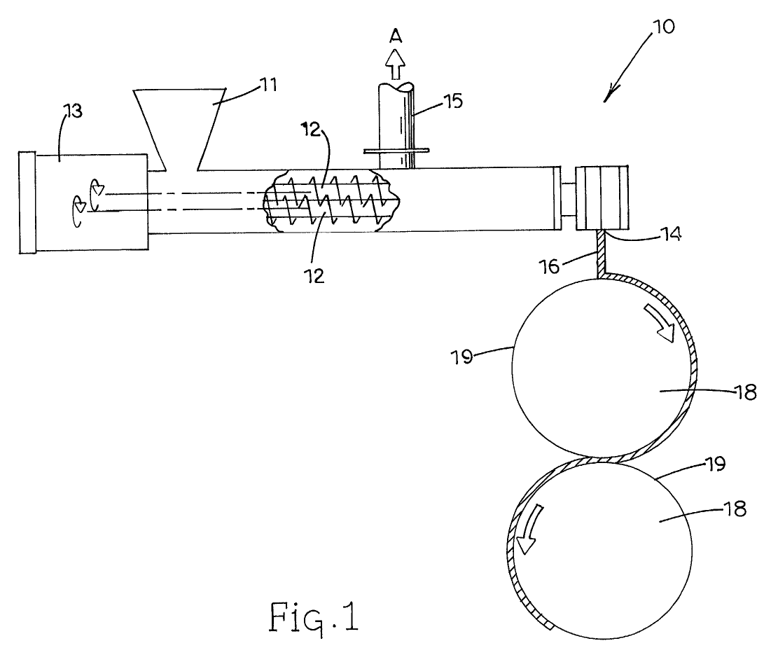

To make the absorbent material, the composition of the thermoplastic polymer binder resin, the SAP particles, and water may be combined using twin screw extrusion mechanism 10, represented in FIG. 1. In contrast, the film casting and blowing process typically employs a single screw extruder. To the best of the inventors' knowledge, twin screw devices are generally used for blending materials and are not combined with film forming operations.

According to the preferred embodiment of the present invention, the thermoplastic polymer binder resin and the SAP are mixed and the mixture is fed to twin screw extrusion mechanism 10 through feed inlet 11 and then compounded with heat and driven by twin screws 12 which are driven by motor 13 toward exit openings 14. Then, the compounded resultant is pushed through exit openings 14 into its final shape. It is believed that the configuration and use of twin screws 12 in an extruder mechanism, combined with the preferred small size of the SAP particles under about 100 μm, helps to enable the resulting absorbent material to have up to about 60 wt % of SAP, and it is contemplated that up to about 90 wt % SAP may be achieved.

During the step of extruding and driving the thermoplastic polymer binder resin/SAP composition toward exit openings 14, excess moisture is drawn off and vented from the composition through at least one vent 15.

Vent 15 is connected to a source of negative pressure (not shown) to create suction in the direction of arrow A for drawing off excess accumulated moisture to maintain the moisture content of the resulting absorbent material in the range from about 0.1 to about 10 wt % water. The presence of moisture significantly greater than this amount could result in the SAP already having absorbed water in sufficient quantity to detract significantly from its absorbency effectiveness in its end use and to interrupt the integrity of the absorbent material.

Exit openings 14 may be of any shape to produce a desired final shape of absorbent material.

In one embodiment, the shape of exit openings 14 is circular so that the extruded absorbent material resembles strands or strings. These strings can then be cut into individual pellets of absorbent material (not shown).

In a preferred embodiment, the shape of exit openings 14 resembles a slot such that the extrudate is planar and takes the shape of self-supporting sheet or film 16. Preferably, film 16 has a thickness of generally from about 1 to about 25 mils (about 0.025 to about 0.625 mm), more preferably about 2 to about 20 mils (about 0.050 to about 0.500 mm), and even more preferably about 5 to about 10 mils (about 0.125 to about 0.250 mm). This enables film 16 to be used in a variety of end applications, such as in a cable as discussed above and below.

As film 16 leaves exit opening 14, a slight expansion in film 16 is noted. This may be a result of the presence of between about 0.1 and about 10 wt % of water in the absorbent material. As film 16 is extruded, some of this water blows off, which may cause the noted thickening of film 16 as it leaves the extrusion die.

As the concentration of SAP in the composition is increased, it is believed that the thickness of film 16 should be increased to offset the decrease in concentration of binder resin and thus to maintain physical integrity of film 16.

Typically, extrudates are quenched by rapid cooling in a chilled water bath to “cure” the output. Such conventional rapid cooling is inappropriate for the present invention because the absorbent material extrudate would absorb the water in the chilled water bath and then be unsuitable for future water-absorbing applications. However, there may still be a need to cure the output with rapid cooling, unless, or course, an injection molded product is desired to be made from the extrudate. Thus, after the absorbent material extrudate of the present invention is pushed through exit opening 14, it may be quenched by non-liquid quenching (particularly non-aqueous quenching), including, but not limited to, cool air, chill rolls, or a cold stainless steel belt.

Preferably, the non-aqueous quenching includes at least one chill roll 18 over which extruded film 16 is passed to quench extruded film 16. Chill roll 18 may suitably be a steel roller that includes cylindrical surface 19, which is cooled by an internal cooling fluid that is circulated though chill roll 18. In another embodiment, the non-aqueous quenching includes a chilled gas, such as air, through which film 16 is passed.

Instead of making film directly by use of a twin screw extruder with slot-shaped exit opening 14, pellets can be cut from oblong strand, string or fiber shapes made from a twin screw extruder with circular-shaped exit openings 14 for sale to customers to extrude their own films, as long as non-liquid, i.e., non-aqueous, quenching is used for post extrusion rapid cooling.

Typically, in the manufacture of films, polymer pellets are brought into a heated area where the polymer feed is melted and heated to its extrusion temperature and extruded as a tubular “blown bubble” through an annular die. Other methods, such as “slot die” extrusion wherein the resultant extrudate is in planar, as opposed to tubular, form are also well known. If heat-shrinkable film is desired, then after extrusion, the film is typically cooled and then reheated and stretched, i.e. oriented by “tenter framing” or by inflating with a “trapped bubble”, to impart the heat-shrinkable property to the film, as is further described below.

More particularly, manufacturing of films may be accomplished as follows. For instance, the manufacture of shrink films may be generally accomplished by extrusion (single layer films) or coextrusion (multi-layer films) of polymeric resins which have been heated to or above their flow or melting point from an extrusion or coextrusion die in, for example, either tubular or planar (sheet) form, followed by a post extrusion cooling. The stretching for orientation may be conducted at some point during the cool down and while the film is still hot and within its orientation temperature range followed by completing the cooling. Alternatively, after the post extrusion cooling, the relatively thick “tape” extrudate is then reheated to a temperature within its orientation temperature range and stretched to orient or to align the crystallites and/or molecules of the heated tape and then cooled.

The temperature range for orientation will vary with the different resinous polymers and/or blends of polymers. However, the orientation temperature range for a given polymer may generally be stated to be below the crystalline melting point of the polymer but above the second order transition temperature (sometimes referred to as the glass transition point) thereof. Within this temperature range, the material may be effectively oriented.

The terms “orientation” or “oriented” are used herein to describe generally the process steps and resultant product characteristics obtained by stretching and immediately cooling a resinous polymeric material which has been heated to a temperature within its orientation temperature range to obtain a heat-shrinkable material. This revises the intermolecular configuration of the material by physical alignment of the crystallites and/or molecules of the material to improve certain mechanical properties of the film such as, for example, shrink tension and orientation release stress. Both of these properties may be measured in accordance with ASTM D 2838-81.

When the stretching force is applied in one direction, monoaxial orientation results. When the stretching force is simultaneously or sequentially applied in two directions, biaxial orientation results. The term oriented is also herein used interchangeably with the term “heat-shrinkable” with these terms designating a material which has been stretched and set by cooling while substantially retaining its stretched dimensions. An oriented (i.e., heat-shrinkable) material will tend to shrink and thus to return to its original unstretched (unextended) dimensions when heated to an appropriate elevated temperature, such as by passing through a hot air tunnel.

Returning to the basic process for manufacturing the film as discussed above, it can be seen that the film, once extruded (or coextruded if it is a multi-layer film) and initially cooled, is then reheated to within its orientation temperature range and oriented by stretching. The stretching to orient may be accomplished in many ways such as, for example, by “trapped bubble” techniques or “tenter framing”. The “tenter framing” technique is also known as the “flat die” technique. These processes are well known to those in the art and refer to orientation procedures whereby the material is stretched in the cross or transverse direction (TD) and/or in the longitudinal or machine direction (MD). Simultaneous biaxial stretching is often employed with the “trapped bubble” technique, whereas sequential biaxial stretching is often employed with the “tenter frame” technique. After being stretched, the film is quickly cooled while substantially retaining its stretched dimensions to cool the film rapidly and thus set or lock-in the oriented molecular configuration.

Of course, if a film having little or no orientation is desired, e.g., non-oriented or non-heat shrinkable film, the film may be formed from a non-orientable material or, if formed from an orientable material may be formed from a tube by using a “trapped bubble” technique commonly known as the “hot blown” technique. In forming a hot blown film, the tube is not cooled initially after extrusion or coextrusion but rather is first stretched by a hot blown bubble essentially immediately after extrusion while the tube is still at an elevated temperature above the orientation temperature range of the material. Thereafter, the film is cooled, by well-known methods. Those of skill in the art are well familiar with this process and the fact that the resulting film, although stretched, has substantially unoriented characteristics, i.e., the film is not heat-shrinkable. Other methods for forming unoriented films are well known. Exemplary, is the method of cast extrusion or cast coextrusion which, likewise is well known to those in the art.

Many other process variations for forming films (both oriented films and unoriented films) are well known to those in the art. For example, conventional pressing, thermoforming or laminating techniques (including corona laminating) may be employed. For instance, multiple layers may be first coextruded with additional layers thereafter being laminated thereon, or two multi-layer tubes may be coextruded with one of the tubes thereafter being laminated onto the other.

For multi-layer films of the present invention, not every layer has to be a composition of SAP+TPBR. One or more layers may be only TPBR. For instance, a 3-layer film may comprise SAP+TPBR/TPBR/SAP+TPBR, and a 2-layer film may comprise SAP+TPBR/TPBR.

The composition of the present invention is sufficiently homogenously blended and formed into an absorbent material such that a film has adequate tensile strength to maintain continuity without developing holes. In the preferred embodiment, the small size of the SAP particles and the use of twin screws in an extruder mechanism both help to ensure that the SAP particles and the TPBR are sufficiently homogenously blended to maintain continuity and to ensure adequate tensile strength.

Typically, the absorbent material of the present invention absorbs liquid to at least about 70% of its maximum capacity within about 15 minutes after exposure to the liquid, particularly for deionized water as the liquid. The maximum capacity in this sense is the amount of liquid that could be absorbed if the same quantity of superabsorbent polymer were present without the thermoplastic polymer binder resin. This improved absorbency enables the absorbent material to be employed in uses in which it is desirable for liquid to be quickly absorbed.

The absorbent material of the present invention also has application in many diverse industries, and thus may be employed for virtually any traditional use for which SAPs are employed, including those uses mentioned above, especially for those uses involving not only absorbency, but also swelling/water blocking and/or controlled release.

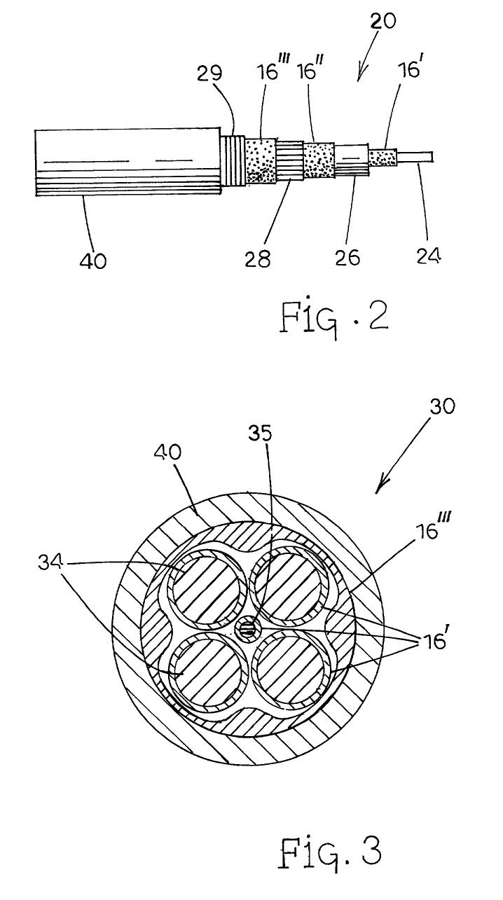

More particularly, FIGS. 2 and 3 illustrate typical uses of preferred embodiments within the cable industry where film 16 is disposed around one or more components of a cable to prevent migration of water within the cable.

FIG. 2 illustrates a typical cable application 20 in which core 24 is wrapped or enveloped with an inner layer of film 16′ formed as described above. Then a layer of inner insulation 26 is disposed around inner layer of film 16′. An intermediate layer of the same type of film 16″ is wrapped around inner insulation 26, and multiconductor 28 is disposed around intermediate layer of film 16″. Multiconductor 28 is then wrapped with an outer layer of the same type of film 16′″, and metallic jacket 29 is disposed around outer layer of film 16′″. Finally, the entire cable assembly is encased in outer insulation 40.

FIG. 3 illustrates another typical cable application 30 in which cable 30 has a plurality of cores 34 and a central longitudinal strength member 35. Each core 34 and also strength member 35 is wrapped or enveloped with an inner layer of film 16′, and the entire bundle of cores 34 is then wrapped with an outer layer of film 16′″ which may expand to conform to the contours within the cable bundle. The entire cable bundle assembly is then encased with outer insulation 40.

Instead of wrapping or enveloping film 16 around the cable components, a tape of film 16 may be placed directly along at least one component in cable 20 or 30 for which liquid migration prevention is to be provided. (Not illustrated.) The absorbent material of the present invention has both water blocking and water absorbing properties. The water blocking properties are exhibited when the absorbent material, illustrated as film 16, is placed into a confining structure, such as those illustrated in FIGS. 2 and 3. When film 16 encounters water, film 16 itself swells to block moisture from passing around film 16. Further, when water touches film 16, a superabsorbent polymer “slime” forms on film 16 surface and this “slime” then fills in capillary gaps in the cable structure and stops the migration of water through these capillary gaps that otherwise provide pathways through which water can travel. It is noted that materials with water blocking properties are not necessarily water absorbent; however, the absorbent material of the present invention exhibits both water blocking and water absorbing properties.

The water absorbing properties of the absorbent material of the present invention not only aid in the water blocking but also are significant in themselves. For example, it is important that the water blocking properties of the absorbent material act quickly to stop the migration of water as rapidly as possible. Thus, it is important for the absorbent material to absorb water quickly to trigger these water blocking properties. The following examples illustrate the absorbency of the absorbent material of the present invention.

### Laboratory Examples

TB. The tea bag test was conducted at ambient conditions of temperature. The retention capacity of the film was determined according to the tea bag test method.

Cut film samples were weighed and each was placed in a tea bag. The tea bags were then sealed and marked for identification. For each test series, 3 samples were prepared for each time interval as indicated in the Examples below.

The prepared samples were submerged in test solution of deionized water. (Other solutions may be employed, such as 0.9% aqueous NaCl.) At the prescribed time intervals, samples were extracted from the test solution and were suspended for 1 minute to allow for surface test solution to drip off. The prepared samples were then weighed.

In addition to the prepared film samples, a set of 3 blank tea bags were prepared and subjected to the same conditions as the film samples. Then, the amount of test solution held by the film sample in the tea bag was determined as follows.

F=Final weight of sample, tea bag and solution absorbed

S=Die cut film sample weight

B=Weight of tea bag blank and solution absorbed

{[(F₁−S₁)+(F₂−S₂)+(F₃−S₃)]/3}−[(B₁+B₂+B₃)/3]=solution absorbed by sample.

Also, tea bag tests were conducted on SAP alone, and calculations repeated, where S was the sample weight of the SAP.

DW. The demand wetability test was conducted at ambient conditions of temperature. The acquisition of the film was determined according to the demand wetability test method.

Cut film samples were weighed and each was made into a sandwich with two filter papers that had also been weighed, one on the top surface of the film sample and one on the bottom surface of the film sample. The two filter papers allowed for wetting of both surfaces of the film during the test. The average absorption of two filter papers was previously determined prior to initiation of the test.

Individually, a sandwich was placed on the platen of a weight scale. The platen was connected via a tube to a reservoir containing test solution. No weight was used on top of the sandwich.

Different test solutions were employed: Dl water, hard water, soft water, or 0.9% aqueous NaCl. Test solution was allowed to flow from the reservoir through the tube to the platen and the sandwich.

At 5 minute intervals, the sandwich was weighed, and the average absorption of the two filter papers subtracted, in order to determine the amount of test solution absorbed by the film sample.

Materials. The following were employed in the Laboratory Examples recited below. EVA with a LMI of 26 was ELVAX 3180 (which has 28% VA) purchased from DuPont. EVA with a HMI of 426 was ESCORENE LD 751.36 (which has 27.8% VA) purchased from Exxon. Na polyacrylate was FAVOR 800 SAB fines with a mean particle size of 50 μm obtained from Stockhausen. The twin screw extruder was a slot die type of extruder, sold as the ZSE-27 modular/multi-mode twin screw extruder. All percentages of materials were weight percentages.

### EXAMPLE I

Using a twin screw extruder with slot-shaped exit openings (as described above vis-a-vis FIG. 1), 4 films (thickness=10 mils) of absorbent material were made. Each film (non-heat-shrinkable) had differing percentages (10%, 30%, 50%, and 60%) of sodium polyacrylate fines (as the particulate SAP) and of EVA (90%, 70%, 50%, and 40%) with a low melt index (as the particulate thermoplastic polymer binder resin), and each was cut into samples. The samples were tested for rate of liquid absorbency of DI water using the above-described TB test.

The following Tables 1-4 summarize results obtained, where each of Samples 1-48 is the average of results for 3 individual samples.

TABLE 1
10% SAP + 90% EVA (Comparison)

| | | Average | Average |
|---|---|---|---|
| | Time | g uptake per | g uptake per |
| Sample | (min) | g sample | g SAP |
| 1 | 5 | 1.26 | 12.61 |
| 2 | 10 | 1.86 | 18.61 |
| 3 | 15 | 1.87 | 18.73 |
| 4 | 20 | 1.67 | 16.69 |
| 5 | 25 | 2.02 | 20.23 |
| 6 | 30 | 0.84 | 8.39 |
| 7 | 60 | 0.73 | 7.25 |
| 8 | 120 | 2.66 | 26.59 |
| 9 | 240 | 4.21 | 42.10 |
| 10 | 840 | 5.55 | 55.46 |
| 11 | 1380 | 5.96 | 59.64 |
| 12 | 2820 | 7.22 | 72.21 |

TABLE 2
30% SAP + 70% EVA

| | | Average | Average |
|---|---|---|---|
| | Time | g uptake per | g uptake per |
| Sample | (min) | g sample | g SAP |
| 13 | 5 | 19.39 | 64.64 |
| 14 | 10 | 25.61 | 85.37 |
| 15 | 15 | 30.21 | 100.69 |
| 16 | 20 | 42.82 | 142.75 |
| 17 | 25 | 46.94 | 156.47 |
| 18 | 30 | 48.23 | 160.78 |
| 19 | 60 | 54.63 | 182.10 |
| 20 | 120 | 63.30 | 210.99 |
| 21 | 240 | 63.04 | 210.15 |
| 22 | 840 | 66.26 | 220.87 |
| 23 | 1380 | 69.54 | 231.80 |
| 24 | 2820 | 71.09 | 236.97 |

TABLE 3
50% SAP + 50% EVA

| | | Average | Average |
|---|---|---|---|
| | Time | g uptake per | g uptake per |
| Sample | (min) | g sample | g SAP |
| 25 | 5 | 45.63 | 91.26 |
| 26 | 10 | 73.00 | 146.01 |
| 27 | 15 | 79.78 | 159.56 |
| 28 | 20 | 91.97 | 183.95 |
| 29 | 25 | 85.22 | 170.45 |
| 30 | 30 | 85.12 | 170.24 |
| 31 | 60 | 95.48 | 190.97 |
| 32 | 120 | 100.86 | 201.73 |
| 33 | 240 | 106.97 | 213.94 |
| 34 | 840 | 111.12 | 222.23 |
| 35 | 1380 | 112.16 | 224.33 |
| 36 | 2820 | 116.28 | 232.56 |

TABLE 4
60% SAP + 40% EVA

| | | Average | Average |
|---|---|---|---|
| | Time | g uptake per | g uptake per |
| Sample | (min) | g sample | g SAP |
| 37 | 5 | 49.17 | 81.94 |
| 38 | 10 | 76.12 | 126.87 |
| 39 | 15 | 98.21 | 163.69 |
| 40 | 20 | 103.09 | 171.82 |
| 41 | 25 | 106.02 | 176.70 |
| 42 | 30 | 97.02 | 161.70 |
| 43 | 60 | 100.73 | 167.89 |
| 44 | 120 | 113.51 | 189.18 |
| 45 | 240 | 118.02 | 196.70 |
| 46 | 840 | 123.26 | 205.43 |
| 47 | 1380 | 132.74 | 221.23 |
| 48 | 2820 | 133.87 | 223.12 |

Table 5 below illustrates the uptake in DI water by 1 gram of the same sodium polyacrylate superabsorbent fines used in Samples 1-48 but alone and thus not immobilized in a thermoplastic polymer binder resin. The values for the fines for the first 30 minutes were not determined because the samples exhibited gel blocking. Pure superabsorbent fines absorb very quickly, but pure superabsorbent fines are impractical for the uses discussed above.

Also, the far right column of Table 5 shows a comparison for the uptake in DI water by 1 gram of the same polyacrylate superabsorbent but having a standard particle size of 400 μm.

TABLE 5
1 gram of SAP

| | | | g Absorbed |
|---|---|---|---|
| | | | by 1 g of |
| | | g Absorbed | Standard |
| | Time | by Fines | Particle Size |
| Example | (min) | (50 μm) | (400 μm) |
| 49 | 5 | 0 (gel block) | 169.3 |
| 50 | 10 | 0 (gel block) | 179.5 |
| 51 | 15 | 0 (gel block) | 183.2 |
| 52 | 20 | 0 (gel block) | 186.9 |
| 53 | 25 | 0 (gel block) | 186.2 |
| 54 | 30 | 0 (gel block) | 186.1 |
| 55 | 60 | 130.5 | 187.0 |
| 56 | 120 | 152.3 | |
| 57 | 240 | 165.2 | |
| 58 | 840 | 179.0 (estimate) | |
| 59 | 1380 | 190.7 | |
| 60 | 2820 | 201.0 | |

The far right column of Table 6 below illustrates the relationship (expressed as a %) between the uptake (data from far right column of each of Tables 1 through 4) to the absorbency of SAP that has a standard particle size of 400 μm (data from far right column of Table 5) at various times. As can be seen, absorbent materials with ≧30% SAP absorb ≧70% of capacity within about 20 minutes exposure to DI water.

TABLE 6
(% Capacity Absorbed for Film with 10% SAP)

| | Average g uptake | g absorbed by | % of capacity |
|---|---|---|---|
| | per g of SAP from | 1 g of 400 | absorbed |
| Time | comparison in | μm size SAP | (Table 1/ |
| (min) | Table 1 | from Table 5 | Table 5) |
| 5 | 12.61 | 169.3 | 7.4 |
| 10 | 18.61 | 179.5 | 10.4 |
| 15 | 18.73 | 183.2 | 10.2 |
| 20 | 16.69 | 186.9 | 8.9 |
| 25 | 20.23 | 186.2 | 10.9 |
| 30 | 8.39 | 186.1 | 4.5 |
| 60 | 7.25 | 187.0 | 3.9 |

(% Capacity Absorbed for Film with 30% SAP)

| | Average g | g absorbed by | % of capacity |
|---|---|---|---|
| | uptake per g | 1 g of 400 | absorbed |
| Time | of SAP from | μm size SAP | (Table 2/ |
| (min) | Table 2 | from Table 5 | Table 5) |
| 5 | 64.64 | 169.3 | 38.2 |
| 10 | 85.37 | 179.5 | 47.6 |
| 15 | 100.69 | 183.2 | 55.0 |
| 20 | 142.75 | 186.9 | 76.4 |
| 25 | 156.47 | 186.2 | 84.0 |
| 30 | 160.78 | 186.1 | 86.4 |
| 60 | 182.10 | 187.0 | 97.4 |

(% Capacity Absorbed for Film with 50% SAP)

| | Average g | g absorbed by | % of capacity |
|---|---|---|---|
| | uptake per g | 1 g of 400 | absorbed |
| Time | of SAP from | μm size SAP | (Table 3/ |
| (min) | Table 3 | from Table 5 | Table 5) |
| 5 | 91.26 | 169.3 | 53.9 |
| 10 | 146.01 | 179.5 | 81.6 |
| 15 | 159.56 | 183.2 | 87.1 |
| 20 | 183.95 | 186.9 | 98.4 |
| 25 | 170.45 | 186.2 | 91.5 |
| 30 | 179.24 | 186.1 | 91.5 |
| 60 | 190.97 | 187.0 | 102.1 |

(% Capacity Absorbed for Film with 60% SAP)

| | Average g uptake | g absorbed by | % of capacity |
|---|---|---|---|
| | per g of SAP | 1 g of 400 | absorbed |
| Time | from Table | μm size SAP | (Table 4/ |
| (min) | 4 (60% SAP) | from Table 5 | Table 5) |
| 5 | 81.94 | 169.3 | 48.4 |
| 10 | 126.87 | 179.5 | 70.7 |
| 15 | 163.69 | 183.2 | 89.4 |
| 20 | 171.82 | 186.9 | 91.9 |
| 25 | 176.70 | 186.2 | 94.9 |
| 30 | 161.70 | 186.1 | 86.9 |
| 60 | 167.89 | 187.0 | 89.8 |

### EXAMPLE II

Additional samples of the 4 films from Example I were tested for acquisition of liquid from a reservoir in accordance with the DW test described above, using 4 different liquids, namely DI water, hard water, soft water, and aqueous solution of 0.9% NaCl.

The following Tables 1A, 1B, 1C, 1D through 4A, 4B, 4C, 4D summarize the results obtained.

TABLE 1A
10% SAP + 90% EVA (Comparison)
Test Solution: DI water; film sample wt = 0.05 g
Abs/min (5 min intervals)
determined by the difference of blank filter paper and filter paper with film
Average absorption of filter paper in DI water is 2.73 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0.00 | 0.00 | 0.00 | 0.00 | 0.00 | 0.00 | 0.00 | 0.00 |
| 5 | 0.07 | 0.17 | −0.04 | 0.03 | 0.06 | 0.06 | 1.15 | 1.15 |
| 10 | 0.02 | 0.01 | 0.02 | 0.02 | 0.02 | 0.08 | 0.35 | 1.5 |
| 15 | 0.01 | 0.02 | 0.02 | 0.02 | 0.02 | 0.09 | 0.35 | 1.85 |
| 20 | 0.02 | 0.01 | 0.02 | 0.04 | 0.02 | 0.12 | 0.45 | 2.3 |
| 25 | 0.01 | 0.01 | 0.01 | 0.02 | 0.01 | 0.13 | 0.25 | 2.55 |
| 30 | 0.01 | 0.01 | 0.06 | 0.01 | 0.02 | 0.15 | 0.45 | 3 |
| 35 | 0.01 | 0.01 | 0.01 | 0.01 | 0.01 | 0.16 | 0.2 | 3.2 |
| 40 | 0.01 | 0.01 | 0 | 0.01 | 0.01 | 0.17 | 0.15 | 3.35 |
| 45 | 0.01 | 0.01 | 0 | 0.02 | 0.01 | 0.18 | 0.2 | 3.55 |
| 50 | 0.01 | 0.01 | 0 | 0.01 | 0.01 | 0.19 | 0.15 | 3.7 |
| 55 | 0.01 | 0.01 | 0 | 0.01 | 0.01 | 0.19 | 0.15 | 3.85 |
| 60 | 0.01 | 0.01 | 0 | 0.01 | 0.01 | 0.20 | 0.15 | 4 |
| 65 | 0.01 | 0.01 | 0 | 0.01 | 0.01 | 0.21 | 0.15 | 4.15 |
| 70 | 0.04 | 0.01 | 0.01 | 0.01 | 0.02 | 0.23 | 0.35 | 4.5 |
| 75 | 0 | 0.01 | 0.01 | 0.01 | 0.01 | 0.23 | 0.15 | 4.65 |
| 80 | 0 | 0 | 0.01 | 0.01 | 0.01 | 0.24 | 0.1 | 4.75 |
| 85 | 0 | 0.01 | 0.04 | 0.01 | 0.02 | 0.25 | 0.3 | 5.05 |
| 90 | 0 | 0.01 | 0 | 0 | 0.00 | 0.26 | 0.05 | 5.1 |
| 95 | 0.01 | 0 | 0 | 0 | 0.00 | 0.26 | 0.05 | 5.15 |
| 100 | 0.01 | 0.01 | 0 | 0 | 0.01 | 0.26 | 0.1 | 5.25 |
| 105 | 0.02 | 0.01 | 0 | 0 | 0.01 | 0.27 | 0.15 | 5.4 |
| 110 | 0.01 | 0.01 | 0 | 0 | 0.01 | 0.28 | 0.1 | 5.5 |
| 115 | 0.01 | 0.01 | 0 | 0 | 0.01 | 0.28 | 0.1 | 5.6 |
| 120 | 0.01 | 0.01 | 0 | 0 | 0.01 | 0.29 | 0.1 | 5.7 |
| 125 | 0.02 | 0.01 | 0 | 0 | 0.01 | 0.29 | 0.15 | 5.85 |
| 130 | 0.01 | 0.01 | 0 | 0 | 0.01 | 0.30 | 0.1 | 5.95 |
| 135 | 0.01 | 0.01 | 0 | 0 | 0.01 | 0.30 | 0.1 | 6.05 |
| 140 | 0.01 | 0.01 | 0 | 0 | 0.01 | 0.31 | 0.1 | 6.15 |
| 145 | 0 | 0.01 | 0 | 0 | 0.00 | 0.31 | 0.05 | 6.2 |
| 150 | 0 | 0.01 | 0 | 0 | 0.00 | 0.31 | 0.05 | 6.25 |

TABLE 1B
10% SAP + 90% EVA (Comparison)
Test Solution: hard water; film sample wt = 0.05 g
Abs/min (5 min intervals)
determined by the difference of blank filter paper and filter paper with film
Average absorption of filter paper in hard water is 2.86 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.4 | 0.21 | 0.18 | −0.56 | 0.06 | 0.06 | 1.15 | 1.15 |
| 10 | 0.01 | 0.01 | 0 | 0.01 | 0.01 | 0.07 | 0.15 | 1.3 |
| 15 | 0.02 | 0.01 | 0 | 0 | 0.01 | 0.07 | 0.15 | 1.45 |
| 20 | 0.15 | 0.01 | 0 | 0.01 | 0.04 | 0.12 | 0.85 | 2.3 |
| 25 | 0.02 | 0 | 0 | 0.01 | 0.01 | 0.12 | 0.15 | 2.45 |
| 30 | 0.02 | 0.01 | 0 | 0.01 | 0.01 | 0.13 | 0.2 | 2.65 |
| 35 | 0.13 | 0.01 | 0 | 0.23 | 0.09 | 0.23 | 1.85 | 4.5 |
| 40 | 0.02 | 0.01 | 0 | 0.01 | 0.01 | 0.24 | 0.2 | 4.7 |
| 45 | 0.15 | 0.18 | 0 | 0.01 | 0.09 | 0.32 | 1.7 | 6.4 |
| 50 | 0.01 | 0 | 0 | 0.01 | 0.01 | 0.33 | 0.1 | 6.5 |
| 55 | 0.02 | 0 | 0 | 0.01 | 0.01 | 0.33 | 0.15 | 6.65 |
| 60 | 0.21 | 0 | 0 | 0 | 0.05 | 0.39 | 1.05 | 7.7 |
| 65 | 0.02 | 0 | 0 | 0 | 0.01 | 0.39 | 0.1 | 7.8 |

TABLE 1C
10% SAP + 90% EVA (Comparison)
Test Solution: soft water; film sample wt = 0.05 g
Abs/min (5 min intervals)
determined by the difference of blank filter paper and filter paper with film
Average absorption of filter paper in DI water is 2.59 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.34 | 0.24 | 0.21 | 0.35 | 0.29 | 0.29 | 5.7 | 5.7 |
| 10 | 0.01 | 0 | 0 | 0 | 0.00 | 0.29 | 0.05 | 5.75 |
| 15 | 0.01 | 0 | 0 | 0.01 | 0.01 | 0.29 | 0.1 | 5.85 |
| 20 | 0.01 | 0 | 0 | 0.01 | 0.01 | 0.30 | 0.1 | 5.95 |
| 25 | 0 | 0.01 | 0 | 0.02 | 0.01 | 0.31 | 0.15 | 6.1 |
| 30 | 0.01 | 0.01 | 0 | 0.22 | 0.06 | 0.37 | 1.2 | 7.3 |
| 35 | 0.01 | 0 | 0 | 0.01 | 0.01 | 0.37 | 0.1 | 7.4 |
| 40 | 0.01 | 0 | 0 | 0.01 | 0.01 | 0.38 | 0.1 | 7.5 |
| 45 | 0.19 | 0 | 0 | 0.01 | 0.05 | 0.43 | 1 | 8.5 |
| 50 | 0.01 | 0 | 0 | 0.02 | 0.01 | 0.43 | 0.15 | 8.65 |
| 55 | 0.01 | 0 | 0.01 | 0.22 | 0.06 | 0.49 | 1.2 | 9.85 |
| 60 | 0.01 | 0 | 0.01 | 0 | 0.01 | 0.50 | 0.1 | 9.95 |
| 65 | 0.01 | 0 | 0.01 | 0.01 | 0.01 | 0.51 | 0.15 | 10.1 |
| 70 | 0.23 | 0 | 0.07 | 0.01 | 0.08 | 0.58 | 1.55 | 11.65 |
| 75 | 0.01 | 0 | 0.01 | 0.02 | 0.01 | 0.59 | 0.2 | 11.85 |
| 80 | 0.01 | 0 | 0.01 | 0.22 | 0.06 | 0.65 | 1.2 | 13.05 |
| 85 | 0.01 | 0 | 0.01 | 0 | 0.01 | 0.66 | 0.1 | 13.15 |
| 90 | 0.01 | 0 | 0.19 | 0.01 | 0.05 | 0.71 | 1.05 | 14.2 |
| 95 | 0.02 | 0 | 0 | 0.01 | 0.01 | 0.72 | 0.15 | 14.35 |
| 100 | 0.21 | 0 | 0 | 0.01 | 0.06 | 0.77 | 1.1 | 15.45 |
| 105 | 0.01 | 0 | 0 | 0.01 | 0.01 | 0.78 | 0.1 | 15.55 |
| 110 | 0.01 | 0 | 0 | 0.2 | 0.05 | 0.83 | 1.05 | 16.6 |
| 115 | 0.01 | 0 | 0 | 0.01 | 0.01 | 0.84 | 0.1 | 16.7 |
| 120 | 0.01 | 0 | 0 | 0.01 | 0.01 | 0.84 | 0 | 16.7 |

TABLE 1D
10% SAP + 90% EVA (Comparison)
Test Solution: 0.9% aqueous NaCl; film sample wt = 0.05 g
Abs/min (5 min intervals)
determined by the difference of blank filter paper and filter paper with film
Average absorption of filter paper in 0.9% aqueous NaCl is 2.60 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.12 | 0.18 | 0.57 | 0.11 | 0.25 | 0.25 | 4.9 | 4.9 |
| 10 | 0.09 | 0.04 | 0.09 | 0.02 | 0.06 | 0.31 | 1.2 | 6.1 |
| 15 | 0.01 | 0.03 | 0.12 | 0.01 | 0.04 | 0.35 | 0.85 | 6.95 |
| 20 | 0.02 | 0.08 | 0.06 | 0.01 | 0.04 | 0.39 | 0.85 | 7.8 |
| 25 | 0.02 | 0.02 | 0.07 | 0.04 | 0.04 | 0.43 | 0.75 | 8.55 |
| 30 | 0.01 | 0.01 | 0.12 | 0 | 0.04 | 0.46 | 0.7 | 9.25 |
| 35 | 0.02 | 0.01 | 0.04 | 0 | 0.02 | 0.48 | 0.35 | 9.6 |
| 40 | 0.02 | 0.01 | 0.06 | 0 | 0.02 | 0.50 | 0.45 | 10.05 |
| 45 | 0.01 | 0.01 | 0.07 | 0 | 0.02 | 0.53 | 0.45 | 10.5 |
| 50 | 0.01 | 0.01 | 0.1 | 0 | 0.03 | 0.56 | 0.6 | 11.1 |
| 55 | 0.01 | 0.01 | 0.05 | 0.01 | 0.02 | 0.58 | 0.4 | 11.5 |
| 60 | 0.01 | 0.01 | 0.05 | 0.01 | 0.02 | 0.60 | 0.4 | 11.9 |
| 65 | 0.01 | 0.01 | 0.04 | 0.01 | 0.02 | 0.61 | 0.35 | 12.25 |
| 70 | 0.01 | 0.01 | 0 | 0.01 | 0.01 | 0.62 | 0.15 | 12.4 |
| 75 | 0.01 | 0.01 | 0 | 0.01 | 0.01 | 0.63 | 0.15 | 12.55 |
| 80 | 0.01 | 0.01 | 0 | 0.04 | 0.02 | 0.64 | 0.3 | 12.85 |
| 85 | 0.01 | 0.01 | 0.01 | 0 | 0.01 | 0.65 | 0.15 | 13 |
| 90 | 0.01 | 0.01 | 0.02 | 0 | 0.01 | 0.66 | 0.2 | 13.2 |
| 95 | 0.01 | 0.01 | 0.01 | 0.01 | 0.01 | 0.67 | 0.2 | 13.4 |
| 100 | 0.01 | 0.01 | 0.01 | 0 | 0.01 | 0.68 | 0.15 | 13.55 |
| 105 | 0 | 0.01 | 0.01 | 0 | 0.01 | 0.68 | 0.1 | 13.65 |
| 110 | 0.01 | 0.01 | 0.01 | 0.01 | 0.01 | 0.69 | 0.2 | 13.85 |
| 115 | 0.01 | 0 | 0.01 | 0 | 0.01 | 0.70 | 0.1 | 13.95 |
| 120 | 0 | 0.01 | 0 | 0 | 0.00 | 0.70 | 0.05 | 14 |
| 125 | 0.01 | 0.01 | 0 | 0 | 0.01 | 0.71 | 0.1 | 14.1 |
| 130 | 0 | 0 | 0 | 0 | 0.00 | 0.71 | 0 | 14.1 |
| 135 | 0.01 | 0.01 | 0.01 | 0.01 | 0.01 | 0.72 | 0.2 | 14.3 |

TABLE 2A
30% SAP + 70% EVA
Test Solution: DI water; film sample wt = 0.06 g
Abs/min (5 min intervals)
determined by the difference of blank filter paper and filter paper with film
Average absorption of filter paper in DI water is 2.73 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0.00 | 0.00 | 0.00 | 0.00 | 0.00 | 0.00 | 0.00 | 0.00 |
| 5 | 1.4 | 0.95 | 1.14 | 0.5 | 1.00 | 1.00 | 16.63 | 16.63 |
| 10 | 0.36 | 0.37 | 0.16 | 0.36 | 0.31 | 1.31 | 5.21 | 21.83 |
| 15 | 0.28 | 0.28 | 0.01 | 0.03 | 0.15 | 1.46 | 2.50 | 24.33 |
| 20 | 0.31 | 0.27 | 0.68 | 0.41 | 0.42 | 1.88 | 6.96 | 31.29 |
| 25 | 0.2 | 0.3 | 0 | 0.01 | 0.13 | 2.01 | 2.13 | 33.42 |
| 30 | 0.19 | 0.21 | 0 | 0.02 | 0.11 | 2.11 | 1.75 | 35.17 |
| 35 | 0.19 | 0.15 | 0.01 | 0.48 | 0.21 | 2.32 | 3.46 | 38.63 |
| 40 | 0.16 | 0.15 | 0.83 | 0.01 | 0.29 | 2.61 | 4.79 | 43.42 |
| 45 | 0.16 | 0.15 | 0 | 0 | 0.08 | 2.68 | 1.29 | 44.71 |
| 50 | 0.15 | 0.18 | 0 | 0.01 | 0.09 | 2.77 | 1.42 | 46.13 |
| 55 | 0.09 | 0.07 | 0 | 0.56 | 0.18 | 2.95 | 3.00 | 49.13 |
| 60 | 0.04 | 0.15 | 0 | 0 | 0.05 | 3.00 | 0.79 | 49.92 |
| 65 | 0.06 | 0.04 | 0 | 0 | 0.03 | 3.02 | 0.42 | 50.33 |
| 70 | 0.1 | 0.17 | 0 | 0 | 0.07 | 3.09 | 1.13 | 51.46 |
| 75 | 0.06 | 0.02 | 0 | 0 | 0.02 | 3.11 | 0.33 | 51.79 |
| 80 | 0.08 | 0.03 | 0 | 0 | 0.03 | 3.14 | 0.46 | 52.25 |
| 85 | 0.02 | 0.06 | 0 | 0 | 0.02 | 3.16 | 0.33 | 52.58 |
| 90 | 0.12 | −0.06 | 0 | 0 | 0.02 | 3.17 | 0.25 | 52.83 |
| 95 | −0.02 | 0.08 | 0 | 0 | 0.02 | 3.19 | 0.25 | 53.08 |
| 100 | 0.01 | 0.1 | 0 | 0 | 0.03 | 3.21 | 0.46 | 53.54 |
| 105 | 0.07 | 0.01 | 0 | 0 | 0.02 | 3.23 | 0.33 | 53.88 |
| 110 | 0 | 0 | 0 | 0 | 0.00 | 3.23 | 0.00 | 53.88 |
| 115 | 0 | 0 | 0 | 0 | 0.00 | 3.23 | 0.00 | 53.88 |
| 120 | 0 | 0 | 0 | 0 | 0.00 | 3.23 | 0.00 | 53.88 |
| 125 | 0 | 0 | 0 | 0 | 0.00 | 3.23 | 0.00 | 53.88 |
| 130 | 0 | 0 | 0 | 0 | 0.00 | 3.23 | 0.00 | 53.88 |
| 135 | 0 | 0 | 0 | 0 | 0.00 | 3.23 | 0.00 | 53.88 |
| 140 | 0 | 0 | 0 | 0 | 0.00 | 3.23 | 0.00 | 53.88 |
| 145 | 0 | 0 | 0 | 0 | 0.00 | 3.23 | 0.00 | 53.88 |
| 150 | 0 | 0 | 0 | 0 | 0.00 | 3.23 | 0.00 | 53.88 |

TABLE 2B
30% SAP + 70% EVA
Test Solution: hard water; film sample wt = 0.06 g
Abs/min (5 min intervals)
determined by the difference of blank filter paper and filter paper with film
Average absorption of filter paper in hard water is 2.86 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.28 | 0.29 | 0.56 | 0.15 | 0.32 | 0.32 | 5.33 | 5.33 |
| 10 | 0 | 0 | 0 | 0 | 0.00 | 0.32 | 0.00 | 5.33 |
| 15 | 0 | 0.01 | 0 | 0 | 0.00 | 0.32 | 0.04 | 5.38 |
| 20 | 0.01 | 0.01 | 0.01 | 0 | 0.01 | 0.33 | 0.13 | 5.50 |
| 25 | 0.01 | 0.01 | 0.01 | 0 | 0.01 | 0.34 | 0.13 | 5.63 |
| 30 | 0.01 | 0.02 | 0.01 | 0 | 0.01 | 0.35 | 0.17 | 5.79 |
| 35 | 0.02 | 0.27 | 0.01 | 0 | 0.08 | 0.42 | 1.25 | 7.04 |
| 40 | 0.24 | 0.01 | 0 | 0 | 0.06 | 0.49 | 1.04 | 8.08 |
| 45 | 0.01 | 0.01 | 0 | 0 | 0.01 | 0.49 | 0.08 | 8.17 |
| 50 | 0.01 | 0.01 | 0 | 0 | 0.01 | 0.50 | 0.08 | 8.25 |
| 55 | 0.01 | 0.02 | 0 | 0.01 | 0.01 | 0.51 | 0.17 | 8.42 |
| 60 | 0.01 | 0.01 | 0.01 | 0.01 | 0.01 | 0.52 | 0.17 | 8.58 |
| 65 | 0.02 | 0.3 | 0.27 | 0 | 0.15 | 0.66 | 2.46 | 11.04 |

TABLE 2C
30% SAP + 70% EVA
Test Solution: soft water; film sample wt = 0.06 g
Abs/min (5 min intervals)
determined by the difference of blank filter paper and filter paper with film
Average absorption of filter paper in soft water is 2.59 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.7 | 0.61 | 0.12 | 0.87 | 0.58 | 0.58 | 9.58 | 9.58 |
| 10 | 0.05 | 0.07 | 0.36 | 0.39 | 0.22 | 0.79 | 3.63 | 13.21 |
| 15 | 0.04 | 0.37 | 0.04 | 0.05 | 0.13 | 0.92 | 2.08 | 15.29 |
| 20 | 0.4 | 0.01 | 0.38 | 0.35 | 0.29 | 1.20 | 4.75 | 20.04 |
| 25 | 0.03 | 0.04 | 0.37 | 0.41 | 0.21 | 1.42 | 3.54 | 23.58 |
| 30 | 0.04 | 0.04 | 0.02 | 0.02 | 0.03 | 1.45 | 0.50 | 24.08 |
| 35 | 0.04 | 0.27 | 0.41 | 0.46 | 0.30 | 1.74 | 4.92 | 29.00 |
| 40 | 0.29 | 0 | 0.03 | 0.02 | 0.09 | 1.83 | 1.42 | 30.42 |
| 45 | 0.01 | 0.02 | 0.48 | 0.5 | 0.25 | 2.08 | 4.21 | 34.63 |
| 50 | 0.02 | 0.04 | 0.02 | 0.02 | 0.03 | 2.10 | 0.42 | 35.04 |
| 55 | 0.04 | 0.04 | 0.54 | 0.54 | 0.29 | 2.39 | 4.83 | 39.88 |
| 60 | 0.04 | 0.25 | 0.02 | 0.01 | 0.08 | 2.47 | 1.33 | 41.21 |
| 65 | 0.28 | 0.04 | 0.58 | 0.04 | 0.24 | 2.71 | 3.92 | 45.13 |
| 70 | 0 | 0.01 | 0.01 | 0.52 | 0.14 | 2.84 | 2.25 | 47.38 |
| 75 | 0.01 | 0.03 | 0.03 | 0.03 | 0.03 | 2.87 | 0.42 | 47.79 |
| 80 | 0.03 | 0.03 | 0.59 | 0.58 | 0.31 | 3.18 | 5.13 | 52.92 |
| 85 | 0.03 | 0.03 | 0.03 | 0.01 | 0.03 | 3.20 | 0.42 | 53.33 |
| 90 | 0.03 | 0.36 | 0.61 | 0.04 | 0.26 | 3.46 | 4.33 | 57.67 |
| 95 | 0.04 | 0 | 0.01 | 0.58 | 0.16 | 3.62 | 2.63 | 60.29 |
| 100 | 0.33 | 0 | 0.04 | 0.01 | 0.10 | 3.71 | 1.58 | 61.88 |
| 105 | 0 | 0.02 | 0.6 | 0.03 | 0.16 | 3.88 | 2.71 | 64.58 |
| 110 | 0.01 | 0.04 | 0.02 | 0.59 | 0.17 | 4.04 | 2.75 | 67.33 |
| 115 | 0.02 | 0.04 | 0.64 | 0.01 | 0.18 | 4.22 | 2.96 | 70.29 |
| 120 | 0.03 | 0.43 | 0.01 | 0.03 | 0.13 | 4.34 | 2.08 | 72.38 |

TABLE 2D
30% SAP + 70% EVA
Test Solution: 0.9% aqueous NaCl; film sample wt = 0.06 g
Abs/min (5 min intervals)
determined by the difference of blank filter paper and filter paper with film
Average absorption of filter paper in 0.9% aqueous NaCl is 2.60 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.76 | 1.05 | 0.57 | 0.58 | 0.74 | 0.74 | 12.33 | 12.33 |
| 10 | 0.13 | 0.05 | 0.07 | 0.06 | 0.08 | 0.82 | 1.29 | 13.63 |
| 15 | 0.03 | 0.03 | 0.06 | 0.05 | 0.04 | 0.86 | 0.71 | 14.33 |
| 20 | 0.02 | 0.04 | 0.06 | 0.08 | 0.05 | 0.91 | 0.83 | 15.17 |
| 25 | 0.03 | 0.03 | 0.05 | 0.02 | 0.03 | 0.94 | 0.54 | 15.71 |
| 30 | 0.03 | 0.02 | 0.04 | 0.02 | 0.03 | 0.97 | 0.46 | 16.17 |
| 35 | 0.03 | 0.03 | 0.03 | 0.03 | 0.03 | 1.00 | 0.50 | 16.67 |
| 40 | 0.03 | 0.03 | 0.02 | 0.03 | 0.03 | 1.03 | 0.46 | 17.13 |
| 45 | 0.03 | 0.03 | 0.03 | 0.03 | 0.03 | 1.06 | 0.50 | 17.63 |
| 50 | 0.04 | 0.03 | 0.02 | 0.03 | 0.03 | 1.09 | 0.50 | 18.13 |
| 55 | 0.05 | 0.03 | 0.02 | 0.02 | 0.03 | 1.12 | 0.50 | 18.63 |
| 60 | 0.01 | 0.03 | 0.02 | 0.03 | 0.02 | 1.14 | 0.38 | 19.00 |
| 65 | 0.01 | 0.03 | 0.02 | 0.06 | 0.03 | 1.17 | 0.50 | 19.50 |
| 70 | 0.01 | 0.03 | 0.02 | 0.01 | 0.02 | 1.19 | 0.29 | 19.79 |
| 75 | 0.01 | 0.02 | 0.02 | 0.01 | 0.02 | 1.20 | 0.25 | 20.04 |
| 80 | 0.01 | 0.01 | 0.02 | 0.01 | 0.01 | 1.22 | 0.21 | 20.25 |
| 85 | 0.02 | 0.01 | 0.01 | 0 | 0.01 | 1.23 | 0.17 | 20.42 |
| 90 | 0.01 | 0.02 | 0.02 | 0 | 0.01 | 1.24 | 0.21 | 20.63 |
| 95 | 0.01 | 0.01 | 0.05 | 0 | 0.02 | 1.26 | 0.29 | 20.92 |
| 100 | 0.05 | 0.02 | 0.01 | 0.01 | 0.02 | 1.28 | 0.38 | 21.29 |
| 105 | 0 | 0.01 | 0.01 | 0.01 | 0.01 | 1.29 | 0.13 | 21.42 |
| 110 | 0 | 0.01 | 0.01 | 0.01 | 0.01 | 1.29 | 0.13 | 21.54 |
| 115 | 0 | 0.01 | 0.01 | 0.01 | 0.01 | 1.30 | 0.13 | 21.67 |
| 120 | 0 | 0.05 | 0.01 | 0.04 | 0.03 | 1.33 | 0.42 | 22.08 |
| 125 | 0 | 0.01 | 0.01 | 0 | 0.01 | 1.33 | 0.08 | 22.17 |
| 130 | 0 | 0.01 | 0 | 0 | 0.00 | 1.33 | 0.04 | 22.21 |
| 135 | 0 | 0.02 | 0.01 | 0 | 0.01 | 1.34 | 0.13 | 22.33 |

TABLE 3A
50% SAP + 50% EVA
Test Solution: DI water; film sample wt = 0.15 g
Abs/min (5 min intervals)
determined by the difference of blank filter paper and filter paper with film
Average absorption of filter paper in DI water is 2.73 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0.00 | 0.00 | 0.00 | 0.00 | 0.00 | 0.00 | 0.00 | 0.00 |
| 5 | 4.40 | 4.00 | 4.33 | 1.23 | 3.49 | 3.49 | 23.27 | 23.27 |
| 10 | 3.04 | 3.57 | 2.58 | 2.99 | 3.05 | 6.54 | 20.30 | 43.57 |
| 15 | 2.01 | 2.03 | 2.12 | 2.73 | 2.22 | 8.76 | 14.82 | 58.38 |
| 20 | 0.85 | 0.82 | 1.56 | 0.53 | 0.94 | 9.70 | 6.27 | 64.65 |
| 25 | 0.01 | 0.77 | 0.07 | 0.59 | 0.36 | 10.06 | 2.40 | 67.05 |
| 30 | 0.00 | 0.01 | 0.00 | 0.00 | 0.00 | 10.06 | 0.02 | 67.07 |
| 35 | 0.00 | 0.01 | 0.00 | 0.00 | 0.00 | 10.06 | 0.02 | 67.08 |
| 40 | 0.00 | 0.01 | 0.00 | 0.00 | 0.00 | 10.07 | 0.02 | 67.10 |
| 45 | 0.00 | 0.00 | 0.00 | 0.00 | 0.00 | 10.07 | 0.00 | 67.10 |
| 50 | 0.00 | 0.01 | 0.00 | 0.00 | 0.00 | 10.07 | 0.02 | 67.12 |
| 55 | 0.00 | 0.01 | 0.00 | 0.00 | 0.00 | 10.07 | 0.02 | 67.13 |
| 60 | 0.00 | 0.39 | 0.00 | 0.00 | 0.10 | 10.17 | 0.65 | 67.78 |
| 65 | 0.00 | 0.00 | 0.00 | 0.00 | 0.00 | 10.17 | 0.00 | 67.78 |
| 70 | 0.00 | 0.00 | 0.00 | 0.00 | 0.00 | 10.17 | 0.00 | 67.78 |
| 75 | 0.00 | 0.00 | 0.00 | 0.00 | 0.00 | 10.17 | 0.00 | 67.78 |
| 80 | 0.00 | 0.00 | 0.00 | 0.00 | 0.00 | 10.17 | 0.00 | 67.78 |
| 85 | 0.00 | 0.00 | 0.00 | 0.00 | 0.00 | 10.17 | 0.00 | 67.78 |
| 90 | 0.00 | 0.00 | 0.00 | 0.00 | 0.00 | 10.17 | 0.00 | 67.78 |
| 95 | 0.00 | 0.00 | 0.00 | 0.00 | 0.00 | 10.17 | 0.00 | 67.78 |
| 100 | 0.00 | 0.00 | 0.00 | 0.00 | 0.00 | 10.17 | 0.00 | 67.78 |
| 105 | 0.00 | 0.00 | 0.00 | 0.00 | 0.00 | 10.17 | 0.00 | 67.78 |
| 110 | 0.00 | 0.00 | 0.00 | 0.00 | 0.00 | 10.17 | 0.00 | 67.78 |
| 115 | 0.00 | 0.00 | 0.00 | 0.00 | 0.00 | 10.17 | 0.00 | 67.78 |
| 120 | 0.00 | 0.00 | 0.00 | 0.00 | 0.00 | 10.17 | 0.00 | 67.78 |
| 125 | 0.00 | 0.00 | 0.00 | 0.00 | 0.00 | 10.17 | 0.00 | 67.78 |
| 130 | 0.00 | 0.00 | 0.00 | 0.00 | 0.00 | 10.17 | 0.00 | 67.78 |
| 135 | 0.00 | 0.00 | 0.00 | 0.00 | 0.00 | 10.17 | 0.00 | 67.78 |
| 140 | 0.00 | 0.00 | 0.00 | 0.00 | 0.00 | 10.17 | 0.00 | 67.78 |
| 145 | 0.00 | 0.00 | 0.00 | 0.00 | 0.00 | 10.17 | 0.00 | 67.78 |
| 150 | 0.00 | 0.00 | 0.00 | 0.00 | 0.00 | 10.17 | 0.00 | 67.78 |

TABLE 3B
50% SAP + 50% EVA
Test Solution: hard water; film sample wt = 0.15 g
Abs/min (5 min intervals)
determined by the difference of blank filter paper and filter paper with film
Average absorption of filter paper in hard water is 2.86 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.15 | 1.06 | 0.74 | 0.12 | 0.52 | 0.52 | 3.45 | 3.45 |
| 10 | 0.43 | 0.04 | 0.48 | 0.41 | 0.34 | 0.86 | 2.27 | 5.72 |
| 15 | 0.58 | 0.5 | 0.51 | 0.3 | 0.47 | 1.33 | 3.15 | 8.87 |
| 20 | 0.02 | 0.03 | 0.01 | 0.03 | 0.02 | 1.35 | 0.15 | 9.02 |
| 25 | 0.37 | 0.74 | 0.02 | 0.38 | 0.38 | 1.73 | 2.52 | 11.53 |
| 30 | 0.09 | 0 | 0.56 | 0.04 | 0.17 | 1.90 | 1.15 | 12.68 |
| 35 | 0.01 | 0.01 | 0 | 0.27 | 0.07 | 1.98 | 0.48 | 13.17 |
| 40 | 0.01 | 0.01 | 0.01 | 0.01 | 0.01 | 1.99 | 0.07 | 13.23 |
| 45 | 0.01 | 0.01 | 0 | 0.02 | 0.01 | 2.00 | 0.07 | 13.30 |
| 50 | 0.01 | 0.02 | 0.01 | 0.25 | 0.07 | 2.07 | 0.48 | 13.78 |
| 55 | 0.01 | 0.39 | 0.02 | 0.01 | 0.11 | 2.18 | 0.72 | 14.50 |
| 60 | 0.29 | 0 | 0.44 | 0 | 0.18 | 2.36 | 1.22 | 15.72 |
| 65 | 0 | 0.01 | 0 | 0.01 | 0.01 | 2.36 | 0.03 | 15.75 |

TABLE 3C
50% SAP + 50% EVA

Test Solution: soft water; film sample wt = 0.15 g
Abs/min (5 min intervals) determined by the difference of
blank filter paper and filter paper with film
Average absorption of filter paper in soft water is 2.59 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 1.55 | 1.67 | 1.18 | 1.71 | 1.53 | 1.53 | 10.18 | 10.18 |
| 10 | 1.28 | 0.99 | 1.54 | 0.65 | 1.12 | 2.64 | 7.43 | 17.62 |
| 15 | 0.82 | 0.98 | 0.05 | 1.01 | 0.72 | 3.36 | 4.77 | 22.38 |
| 20 | 0.62 | 0.72 | 0.76 | 0.49 | 0.65 | 4.01 | 4.32 | 26.70 |
| 25 | 0.58 | 0.52 | 0.57 | 0.54 | 0.55 | 4.56 | 3.68 | 30.38 |
| 30 | 0.41 | 0.05 | 0.05 | 0.01 | 0.13 | 4.69 | 0.87 | 31.25 |
| 35 | 0.08 | 0.45 | 0.01 | 0.01 | 0.14 | 4.83 | 0.92 | 32.17 |
| 40 | 0.39 | 0.01 | 0.01 | 0.02 | 0.11 | 4.93 | 0.72 | 32.88 |
| 45 | 0.06 | 0.48 | 0.01 | 0.02 | 0.14 | 5.08 | 0.95 | 33.83 |
| 50 | 0.07 | 0.02 | 0.01 | 0.01 | 0.03 | 5.10 | 0.18 | 34.02 |
| 55 | 0.4 | 0.04 | 0.01 | 0.01 | 0.12 | 5.22 | 0.77 | 34.78 |
| 60 | 0.05 | 0.36 | 0.01 | 0.49 | 0.23 | 5.45 | 1.52 | 36.30 |
| 65 | 0.06 | 0.05 | 0.52 | 0.01 | 0.16 | 5.61 | 1.07 | 37.37 |
| 70 | 0.49 | 0.6 | 0.01 | 0.01 | 0.28 | 5.88 | 1.85 | 39.22 |
| 75 | 0.03 | 0 | 0 | 0.01 | 0.01 | 5.89 | 0.07 | 39.28 |
| 80 | 0.06 | 0 | 0.01 | 0.01 | 0.02 | 5.91 | 0.13 | 39.42 |
| 85 | 0.56 | 0 | 0 | 0.01 | 0.14 | 6.06 | 0.95 | 40.37 |
| 90 | 0 | 0 | 0 | 0.01 | 0.00 | 6.06 | 0.02 | 40.38 |
| 95 | 0.02 | 0 | 0 | 0.02 | 0.01 | 6.07 | 0.07 | 40.45 |
| 100 | 0.05 | 0 | 0 | 0.01 | 0.02 | 6.08 | 0.10 | 40.55 |
| 105 | 0.04 | 0 | 0 | 0.01 | 0.01 | 6.10 | 0.08 | 40.63 |
| 110 | 0.58 | 0 | 0 | 0 | 0.15 | 6.24 | 0.97 | 41.60 |
| 115 | 0 | 0 | 0 | 0 | 0.00 | 6.24 | 0.00 | 41.60 |
| 120 | 0.03 | 0 | 0 | 0.01 | 0.01 | 6.25 | 0.07 | 41.67 |

TABLE 3D
50% SAP + 50% EVA

Test Solution: 0.9% aqueous NaCl; film sample wt = 0.15 g
Abs/min (5 min intervals) determined by the difference of blank
filter paper and filter paper with film Average absorption of
filter paper in 0.9% aqueous NaCl is 2.60 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 1.8 | 1.24 | 1.5 | 1.32 | 1.47 | 1.47 | 9.77 | 9.77 |
| 10 | 0.67 | 0.48 | 0.56 | 0.57 | 0.57 | 2.04 | 3.80 | 13.57 |
| 15 | 0.38 | 0.35 | 0.45 | 0.4 | 0.40 | 2.43 | 2.63 | 16.20 |
| 20 | 0.26 | 0.31 | 0.35 | 0.22 | 0.29 | 2.72 | 1.90 | 18.10 |
| 25 | 0.22 | 0.24 | 0.19 | 0.18 | 0.21 | 2.92 | 1.38 | 19.48 |
| 30 | 0.04 | 0.08 | 0.13 | 0.03 | 0.07 | 2.99 | 0.47 | 19.95 |
| 35 | 0.04 | 0.06 | 0.1 | 0.03 | 0.06 | 3.05 | 0.38 | 20.33 |
| 40 | 0.03 | 0.02 | 0.04 | 0.04 | 0.03 | 3.08 | 0.22 | 20.55 |
| 45 | 0.01 | 0.05 | 0.06 | 0.04 | 0.04 | 3.12 | 0.27 | 20.82 |
| 50 | 0.02 | 0.04 | 0.08 | 0.07 | 0.05 | 3.18 | 0.35 | 21.17 |
| 55 | 0.02 | 0.02 | 0.01 | 0.01 | 0.02 | 3.19 | 0.10 | 21.27 |
| 60 | 0.03 | 0.03 | 0.01 | 0 | 0.02 | 3.21 | 0.12 | 21.38 |
| 65 | 0.02 | 0.03 | 0 | 0 | 0.01 | 3.22 | 0.08 | 21.47 |
| 70 | 0.02 | 0.03 | 0 | 0 | 0.01 | 3.23 | 0.08 | 21.55 |
| 75 | 0.02 | 0.03 | 0.01 | 0.01 | 0.02 | 3.25 | 0.12 | 21.67 |
| 80 | 0.01 | 0.03 | 0.01 | 0.01 | 0.02 | 3.27 | 0.10 | 21.77 |
| 85 | 0.02 | 0.04 | 0.01 | 0.01 | 0.02 | 3.29 | 0.13 | 21.90 |
| 90 | 0.01 | 0.05 | 0.01 | 0.01 | 0.02 | 3.31 | 0.13 | 22.03 |
| 95 | 0.01 | 0.05 | 0.02 | 0.02 | 0.03 | 3.33 | 0.17 | 22.20 |
| 100 | 0.02 | 0.04 | 0.02 | 0.02 | 0.03 | 3.36 | 0.17 | 22.37 |
| 105 | 0.02 | 0.08 | 0.02 | 0.02 | 0.04 | 3.39 | 0.23 | 22.60 |
| 110 | 0.02 | 0.04 | 0.02 | 0.06 | 0.04 | 3.43 | 0.23 | 22.83 |
| 115 | 0.01 | 0.03 | 0.02 | 0.01 | 0.02 | 3.44 | 0.12 | 22.95 |
| 120 | 0.03 | 0.03 | 0.06 | 0 | 0.03 | 3.47 | 0.20 | 23.15 |
| 125 | 0.03 | 0.02 | 0 | 0 | 0.01 | 3.49 | 0.08 | 23.23 |
| 130 | 0.02 | 0.03 | 0.01 | 0 | 0.02 | 3.50 | 0.10 | 23.33 |
| 135 | 0.02 | 0.01 | 0.01 | 0 | 0.01 | 3.51 | 0.07 | 23.40 |

TABLE 4A
60% SAP + 40% EVA

Test Solution: Dl water; film sample wt = 0.22 g
Abs/min (5 min intervals) determined by the difference
of blank filter paper and filter paper with film
Average absorption of filter paper in Dl water is 2.73 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0.00 | 0.00 | 0.00 | 0.00 | 0.00 | 0.00 | 0.00 | 0.00 |
| 5 | 5.43 | 5.20 | 5.31 | 6.14 | 5.52 | 5.52 | 25.09 | 25.09 |
| 10 | 4.03 | 4.12 | 4.32 | 4.17 | 4.16 | 9.68 | 18.91 | 44.00 |
| 15 | 3.29 | 3.44 | 3.59 | 4.05 | 3.59 | 13.27 | 16.33 | 60.33 |
| 20 | 2.21 | 2.00 | 2.26 | 2.51 | 2.25 | 15.52 | 10.20 | 70.53 |
| 25 | 0.97 | 1.12 | 0.91 | 0.01 | 0.75 | 16.27 | 3.42 | 73.95 |
| 30 | 0.67 | 0.02 | 0.01 | 0.01 | 0.18 | 16.45 | 0.81 | 74.76 |
| 35 | 0.00 | 1.03 | 0.00 | 0.00 | 0.26 | 16.71 | 1.17 | 75.93 |
| 40 | 0.00 | 0.00 | 0.00 | 0.00 | 0.00 | 16.71 | 0.00 | 75.93 |
| 45 | 0.00 | 0.00 | 0.00 | 0.01 | 0.00 | 16.71 | 0.01 | 75.94 |
| 50 | 0.00 | 0.00 | 0.00 | 0.00 | 0.00 | 16.71 | 0.00 | 75.94 |
| 55 | 0.00 | 0.00 | 0.00 | 0.00 | 0.00 | 16.71 | 0.00 | 75.94 |
| 60 | 0.00 | 0.00 | 0.00 | 0.00 | 0.00 | 16.71 | 0.00 | 75.94 |
| 65 | 0.00 | 0.00 | 0.00 | 0.00 | 0.00 | 16.71 | 0.00 | 75.94 |
| 70 | 0.00 | 0.00 | 0.00 | 0.00 | 0.00 | 16.71 | 0.00 | 75.94 |
| 75 | 0.00 | 0.00 | 0.00 | 0.00 | 0.00 | 16.71 | 0.00 | 75.94 |
| 80 | 0.00 | 0.00 | 0.00 | 0.00 | 0.00 | 16.71 | 0.00 | 75.94 |
| 85 | 0.00 | 0.00 | 0.00 | 0.00 | 0.00 | 16.71 | 0.00 | 75.94 |
| 90 | 0.00 | 0.00 | 0.00 | 0.00 | 0.00 | 16.71 | 0.00 | 75.94 |
| 95 | 0.00 | 0.00 | 0.00 | 0.00 | 0.00 | 16.71 | 0.00 | 75.94 |
| 100 | 0.00 | 0.00 | 0.00 | 0.00 | 0.00 | 16.71 | 0.00 | 75.94 |
| 105 | 0.00 | 0.00 | 0.00 | 0.00 | 0.00 | 16.71 | 0.00 | 75.94 |
| 110 | 0.00 | 0.00 | 0.00 | 0.00 | 0.00 | 16.71 | 0.00 | 75.94 |
| 115 | 0.00 | 0.00 | 0.00 | 0.00 | 0.00 | 16.71 | 0.00 | 75.94 |
| 120 | 0.00 | 0.00 | 0.00 | 0.00 | 0.00 | 16.71 | 0.00 | 75.94 |
| 125 | 0.00 | 0.00 | 0.00 | 0.00 | 0.00 | 16.71 | 0.00 | 75.94 |
| 130 | 0.00 | 0.00 | 0.00 | 0.00 | 0.00 | 16.71 | 0.00 | 75.94 |
| 135 | 0.00 | 0.00 | 0.00 | 0.00 | 0.00 | 16.71 | 0.00 | 75.94 |
| 140 | 0.00 | 0.00 | 0.00 | 0.00 | 0.00 | 16.71 | 0.00 | 75.94 |
| 145 | 0.00 | 0.00 | 0.00 | 0.00 | 0.00 | 16.71 | 0.00 | 75.94 |
| 150 | 0.00 | 0.00 | 0.00 | 0.00 | 0.00 | 16.71 | 0.00 | 75.94 |

TABLE 4B
60% SAP + 40% EVA

Test Solution: hard water; film sample wt = 0.22 g
Abs/min (5 min intervals) determined by the difference
of blank filter paper and filter paper with film
Average absorption of filter paper in hard water is 2.86 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0.00 | 0.00 | 0.00 | 0.00 |
| 5 | 0.28 | 0.29 | 0.56 | 0.15 | 0.32 | 0.32 | 1.45 | 1.45 |
| 10 | 0.4 | 0.36 | 0.4 | 0.39 | 0.39 | 0.71 | 1.76 | 3.22 |
| 15 | 0.4 | 0.24 | 0.28 | 0.27 | 0.30 | 1.01 | 1.35 | 4.57 |
| 20 | 0.03 | 0.33 | 0.03 | 0.03 | 0.11 | 1.11 | 0.48 | 5.05 |
| 25 | 0.39 | 0.21 | 0.37 | 0.35 | 0.33 | 1.44 | 1.50 | 6.55 |
| 30 | 0.44 | 0.34 | 0.3 | 0.37 | 0.36 | 1.80 | 1.65 | 8.19 |
| 35 | 0.01 | 0.03 | 0.02 | 0.02 | 0.02 | 1.82 | 0.09 | 8.28 |
| 40 | 0.02 | 0.25 | 0.4 | 0.03 | 0.18 | 2.00 | 0.80 | 9.08 |
| 45 | 0.27 | 0.35 | 0.03 | 0.35 | 0.25 | 2.25 | 1.14 | 10.22 |
| 50 | 0.01 | 0.02 | 0.28 | 0.01 | 0.08 | 2.33 | 0.36 | 10.58 |
| 55 | 0.01 | 0.02 | 0.01 | 0.02 | 0.02 | 2.34 | 0.07 | 10.65 |
| 60 | 0.02 | 0.21 | 0.01 | 0.01 | 0.06 | 2.41 | 0.28 | 10.93 |
| 65 | 0.27 | 0.01 | 0.01 | 0.2 | 0.12 | 2.53 | 0.56 | 11.49 |

TABLE 4C
60% SAP + 40% EVA

Test Solution: soft water; film sample wt = 0.22 g
Abs/min (5 min intervals) determined by the difference
of blank filter paper and filter paper with film
Average absorption of filter paper in soft water is 2.59 g

| | | | | | | Avg Abs (g/g) | |
|---|---|---|---|---|---|---|---|
| Time | Abs/ | Absorption (g) | | Avg Abs (g) | | | Total |
| (min) | min | Abs/min | Abs/min | Period | Total | g/g/min | g/g |

| | | | | | | | |
|---|---|---|---|---|---|---|---|
| 0 | 0 | 0 | 0 | 0 | 0 | 0 | 0 |
| 5 | 3.47 | 3.71 | 2.88 | 3.35 | 3.35 | 15.24 | 15.24 |
| 10 | 1.34 | 1.33 | 1.43 | 1.37 | 4.72 | 6.21 | 21.45 |
| 15 | 1.28 | 1.14 | 1.12 | 1.18 | 5.90 | 5.36 | 26.82 |
| 20 | 1 | 1 | 1.03 | 1.01 | 6.91 | 4.59 | 31.41 |
| 25 | 0.52 | 0.36 | 0.57 | 0.48 | 7.39 | 2.20 | 33.61 |
| 30 | 0.27 | 0.26 | 0.38 | 0.30 | 7.70 | 1.38 | 34.98 |
| 35 | 0.2 | 0.26 | 0.28 | 0.25 | 7.94 | 1.12 | 36.11 |
| 40 | 0.09 | 0.06 | 0.11 | 0.09 | 8.03 | 0.39 | 36.50 |
| 45 | 0.16 | 0.05 | 0.11 | 0.11 | 8.14 | 0.48 | 36.98 |
| 50 | 0.03 | 0.05 | 0.08 | 0.05 | 8.19 | 0.24 | 37.23 |
| 55 | 0.03 | 0.05 | 0.06 | 0.05 | 8.24 | 0.21 | 37.44 |
| 60 | 0.03 | 0.05 | 0.06 | 0.05 | 8.28 | 0.21 | 37.65 |
| 65 | 0.04 | 0.04 | 0.05 | 0.04 | 8.33 | 0.20 | 37.85 |
| 70 | 0.16 | 0.04 | 0.04 | 0.08 | 8.41 | 0.36 | 38.21 |
| 75 | 0.02 | 0.04 | 0.04 | 0.03 | 8.44 | 0.15 | 38.36 |
| 80 | 0.02 | 0.04 | 0.03 | 0.03 | 8.47 | 0.14 | 38.50 |
| 85 | 0.02 | 0.06 | 0.03 | 0.04 | 8.51 | 0.17 | 38.67 |
| 90 | 0.02 | 0.12 | 0.03 | 0.06 | 8.56 | 0.26 | 38.92 |
| 95 | 0.02 | 0 | 0.03 | 0.02 | 8.58 | 0.08 | 39.00 |
| 100 | 0.02 | 0.02 | 0.03 | 0.02 | 8.60 | 0.11 | 39.11 |
| 105 | 0.02 | 0.01 | 0.04 | 0.02 | 8.63 | 0.11 | 39.21 |
| 110 | 0.02 | 0.02 | 0.05 | 0.03 | 8.66 | 0.14 | 39.35 |
| 115 | 0.02 | 0.04 | 0.05 | 0.04 | 8.69 | 0.17 | 39.52 |
| 120 | 0.02 | 0.04 | 0.04 | 0.03 | 8.73 | 0.15 | 39.67 |

TABLE 4D
60% SAP + 40% EVA

Test Solution: 0.9% aqueous NaCl; film sample wt = 0.21 g
Abs/min (5 min intervals) determined by the difference of blank
filter paper and filter paper with film
Average absorption of filter paper in 0.9% aqueous NaCl is 2.60 g

| | | | | | | | Avg Abs (g/g) | |
|---|---|---|---|---|---|---|---|---|
| Time | | Absorption (g) | | | Avg Abs (g) | | | Total |
| (min) | Abs/min | Abs/min | Abs/min | Abs/min | Period | Total | g/g/min | g/g |
| 0 | 0 | 0 | 0 | 0 | 0 | 0 | 0 | 0 |
| 5 | 1.5 | 1.5 | 1.43 | 1.38 | 1.45 | 1.45 | 6.92 | 6.92 |
| 10 | 0.54 | 0.31 | 0.47 | 0.47 | 0.45 | 1.90 | 2.13 | 9.05 |
| 15 | 0.31 | 0.34 | 0.42 | 0.3 | 0.34 | 2.24 | 1.63 | 10.68 |
| 20 | 0.36 | 0.3 | 0.33 | 0.28 | 0.32 | 2.56 | 1.51 | 12.19 |
| 25 | 0.32 | 0.33 | 0.29 | 0.26 | 0.30 | 2.86 | 1.43 | 13.62 |
| 30 | 0.3 | 0.22 | 0.2 | 0.25 | 0.24 | 3.10 | 1.15 | 14.77 |
| 35 | 0.12 | 0.08 | 0.15 | 0.22 | 0.14 | 3.25 | 0.68 | 15.45 |
| 40 | 0.12 | 0.15 | 0.08 | 0.06 | 0.10 | 3.35 | 0.49 | 15.94 |
| 45 | 0.09 | 0.04 | 0.14 | 0.08 | 0.09 | 3.44 | 0.42 | 16.36 |
| 50 | 0.09 | 0.07 | 0.01 | 0.14 | 0.08 | 3.51 | 0.37 | 16.73 |
| 55 | 0.02 | 0.1 | 0.01 | 0.03 | 0.04 | 3.55 | 0.19 | 16.92 |
| 60 | 0.04 | 0.01 | 0.03 | 0.03 | 0.03 | 3.58 | 0.13 | 17.05 |
| 65 | 0.09 | 0.02 | 0.03 | 0.03 | 0.04 | 3.62 | 0.20 | 17.25 |
| 70 | 0.01 | 0.02 | 0.03 | 0.03 | 0.02 | 3.65 | 0.11 | 17.36 |
| 75 | 0.01 | 0.03 | 0.02 | 0.02 | 0.02 | 3.67 | 0.10 | 17.45 |
| 80 | 0.01 | 0.03 | 0.07 | 0.03 | 0.04 | 3.70 | 0.17 | 17.62 |
| 85 | 0.01 | 0.03 | 0 | 0.02 | 0.02 | 3.72 | 0.07 | 17.69 |
| 90 | 0.02 | 0.06 | 0 | 0.02 | 0.03 | 3.74 | 0.12 | 17.81 |
| 95 | 0.02 | 0 | 0 | 0.02 | 0.01 | 3.75 | 0.05 | 17.86 |
| 100 | 0.02 | 0 | 0.01 | 0.02 | 0.01 | 3.76 | 0.06 | 17.92 |
| 105 | 0.08 | 0.01 | 0.01 | 0.02 | 0.03 | 3.79 | 0.14 | 18.06 |
| 110 | 0 | 0 | 0.01 | 0.01 | 0.01 | 3.80 | 0.02 | 18.08 |
| 115 | 0 | 0.01 | 0.01 | 0.02 | 0.01 | 3.81 | 0.05 | 18.13 |
| 120 | 0 | 0.01 | 0 | 0.02 | 0.01 | 3.82 | 0.04 | 18.17 |
| 125 | 0 | 0.01 | 0 | 0.02 | 0.01 | 3.82 | 0.04 | 18.20 |
| 130 | 0 | 0.01 | 0.01 | 0.02 | 0.01 | 3.83 | 0.05 | 18.25 |
| 135 | 0 | 0.02 | 0.01 | 0.02 | 0.01 | 3.85 | 0.06 | 18.31 |

As can be seen from Tables 2A, 2B, 2C, 2D, 3A, 3B, 3C, 3D, 4A, 4B, 4C, and 4D, absorbent materials with ≧30% SAP exhibit very satisfactory demand wetability characteristics.

### EXAMPLE III

Using a twin-screw extruder with slot-shaped exit openings (as described above vis-a-vis FIG. 1), 4 films of absorbent material were made, each containing the same 50% of sodium polyacrylate fines having a particle size of 50 μm and containing the same 50% of EVA.

A first and a second film were made, each with a 10 mil (0.25 mm) thickness. The difference was that the first film had an EVA with a HMI of 426, whereas the second film had an EVA with a LMI of 26.

Then a third and a fourth film were made the same way, but this time each with a 20 mil (0.50 mm) thickness, where the third film had an EVA with a HMI of 426 and the fourth film had an EVA with a LMI of 26.

The 4 films were cut into samples and tested using the above-described TB test. The results are summarized below in Table A for the 10 mil film samples and in Table B for the 20 mil film samples.

TABLE A
(10 mil thickness films)
EVA with HMI
5 minutes

| Sample | Film (g) | Wet (g) | Absorption (g/g) |
|---|---|---|---|
| 1 | 0.20 | 7.82 | 33.87 |
| 2 | 0.20 | 8.93 | 39.20 |
| 3 | 0.20 | 6.31 | 26.64 |
| | | | Avg Abs (g/g) |
| | | | 33.23 |
| Blank 1 | 0.26 | 0.85 | |
| Blank 2 | 0.25 | 0.78 | |
| Average | | 0.81 | |

EVA with LMI
5 minutes

| Sample | Film (g) | Wet (g) | Absorption (g/g) |
|---|---|---|---|
| 1 | 0.20 | 3.21 | 11.18 |
| 2 | 0.20 | 2.88 | 9.47 |
| 3 | 0.20 | 3.07 | 10.30 |
| | | | Avg Abs (g/g) |
| | | | 10.32 |
| Blank 1 | 0.26 | 0.85 | |
| Blank 2 | 0.25 | 0.78 | |
| Average | | 0.81 | |

EVA with HMI
10 minutes

| Sample | Film (g) | Wet (g) | Absorption (g/g) |
|---|---|---|---|
| 1 | 0.20 | 10.23 | 45.21 |
| 2 | 0.20 | 9.82 | 42.51 |
| 3 | 0.20 | 11.03 | 49.27 |
| | | | Avg Abs (g/g) |
| | | | 45.66 |
| Blank 1 | 0.25 | 1.08 | |
| Blank 2 | 0.25 | 0.99 | |
| Average | | 1.03 | |

EVA with LMI
10 minutes

| Sample | Film (g) | Wet (g) | Absorption (g/g) |
|---|---|---|---|
| 1 | 0.2 | 3.97 | 13.83 |
| 2 | 0.2 | 3.55 | 11.61 |
| 3 | 0.2 | 3.42 | 10.93 |
| | | | Avg Abs (g/g) |
| | | | 12.13 |
| Blank 1 | 0.25 | 1.08 | |
| Blank 2 | 0.25 | 0.99 | |
| Average | | 1.03 | |

EVA with HMI
15 minutes

| Sample | Film (g) | Wet (g) | Absorption (g/g) |
|---|---|---|---|
| 1 | 0.20 | 15.32 | 69.49 |
| 2 | 0.20 | 14.75 | 66.26 |
| 3 | 0.20 | 15.08 | 66.26 |
| | | | Avg Abs (g/g) |
| | | | 67.34 |
| Blank 1 | 0.23 | 1.43 | |
| Blank 2 | 0.23 | 1.43 | |
| Average | | 1.43 | |

EVA with LMI
15 minutes

| Sample | Film (g) | Wet (g) | Absorption (g/g) |
|---|---|---|---|
| 1 | 0.20 | 4.75 | 15.37 |
| 2 | 0.20 | 4.73 | 15.58 |
| 3 | 0.20 | 4.37 | 14.08 |
| | | | Avg Abs (g/g) |
| | | | 15.01 |
| Blank 1 | 0.23 | 1.43 | |
| Blank 2 | 0.23 | 1.43 | |
| Average | | 1.43 | |

EVA with HMI
20 minutes

| Sample | Film (g) | Wet (g) | Absorption (g/g) |
|---|---|---|---|
| 1 | 0.20 | 12.65 | 57.21 |
| 2 | 0.20 | 13.17 | 58.94 |
| 3 | 0.20 | 11.85 | 52.95 |
| | | | Avg Abs (g/g) |
| | | | 56.37 |
| Blank 1 | 0.25 | 1.13 | |
| Blank 2 | 0.25 | 1.00 | |
| Average | | 1.06 | |

EVA with LMI
20 minutes

| Sample | Film (g) | Wet (g) | Absorption (g/g) |
|---|---|---|---|
| 1 | 0.20 | 3.71 | 12.29 |
| 2 | 0.20 | 4.65 | 16.78 |
| 3 | 0.20 | 4.79 | 17.36 |
| | | | Avg Abs (g/g) |
| | | | 15.48 |
| Blank 1 | 0.25 | 1.13 | |
| Blank 2 | 0.25 | 1.00 | |
| Average | | 1.06 | |

EVA with HMI
25 minutes

| Sample | Film (g) | Wet (g) | Absorption (g/g) |
|---|---|---|---|
| 1 | 0.20 | 13.07 | 58.42 |
| 2 | 0.20 | 13.03 | 47.62 |
| 3 | 0.20 | 13.19 | 59.91 |
| | | | Avg Abs (g/g) |
| | | | 58.65 |
| Blank 1 | 0.25 | 1.16 | |
| Blank 2 | 0.25 | 1.22 | |
| Average | | 1.19 | |

EVA with LMI
25 minutes

| Sample | Film (g) | Wet (g) | Absorption (g/g) |
|---|---|---|---|
| 1 | 0.20 | 5.40 | 20.06 |
| 2 | 0.20 | 3.93 | 12.72 |
| 3 | 0.20 | 3.91 | 12.62 |
| | | | Avg Abs (g/g) |
| | | | 15.13 |
| Blank 1 | 0.25 | 1.16 | |
| Blank 2 | 0.25 | 1.22 | |
| Average | | 1.19 | |

EVA with HMI
30 minutes

| Sample | Film (g) | Wet (g) | Absorption (g/g) |
|---|---|---|---|
| 1 | 0.20 | 13.61 | 59.73 |
| 2 | 0.20 | 13.56 | 60.08 |
| 3 | 0.20 | 13.16 | 57.51 |
| | | | Avg Abs (g/g) |
| | | | 59.11 |
| Blank 1 | 0.25 | 1.38 | |
| Blank 2 | 0.25 | 1.06 | |
| Average | | 1.22 | |

EVA with LMI
30 minutes

| Sample | Film (g) | Wet (g) | Absorption (g/g) |
|---|---|---|---|
| 1 | 0.20 | 4.68 | 16.31 |
| 2 | 0.20 | 5.28 | 19.38 |
| 3 | 0.20 | 5.27 | 19.06 |
| | | | Avg Abs (g/g) |
| | | | 18.25 |
| Blank 1 | 0.25 | 1.38 | |
| Blank 2 | 0.25 | 1.06 | |
| Average | | 1.22 | |

EVA With HMI
60 minutes

| Sample | Film (g) | Wet (g) | Absorption (g/g) |
|---|---|---|---|
| 1 | 0.20 | 16.56 | 75.05 |
| 2 | 0.20 | 16.03 | 73.98 |
| 3 | 0.20 | 17.17 | 79.33 |
| | | | Avg Abs (g/g) |
| | | | 76.12 |
| Blank 1 | 0.24 | 1.35 | |
| Blank 2 | 0.24 | 1.21 | |
| Average | | 1.28 | |

EVA with LMI
60 minutes

| Sample | Film (g) | Wet (g) | Absorption (g/g) |
|---|---|---|---|
| 1 | 0.20 | 4.92 | 16.97 |
| 2 | 0.20 | 5.38 | 19.74 |
| 3 | 0.20 | 5.69 | 20.96 |
| | | | Avg Abs (g/g) |
| | | | 19.22 |
| Blank 1 | 0.24 | 1.35 | |
| Blank 2 | 0.24 | 1.21 | |
| Average | | 1.28 | |

EVA with HMI
120 minutes

| Sample | Film (g) | Wet (g) | Absorption (g/g) |
|---|---|---|---|
| 1 | 0.20 | 16.80 | 76.12 |
| 2 | 0.20 | 16.75 | 73.98 |
| 3 | 0.20 | 16.56 | 75.65 |
| | | | Avg Abs (g/g) |
| | | | 75.25 |
| Blank 1 | 0.24 | 1.40 | |
| Blank 2 | 0.24 | 1.67 | |
| Average | | 1.53 | |

EVA with LMI
120 minutes

| Sample | Film (g) | Wet (g) | Absorption (g/g) |
|---|---|---|---|
| 1 | 0.20 | 6.13 | 22.00 |
| 2 | 0.20 | 6.04 | 21.19 |
| 3 | 0.20 | 5.88 | 20.82 |
| | | | Avg Abs (g/g) |
| | | | 21.33 |
| Blank 1 | 0.24 | 1.40 | |
| Blank 2 | 0.24 | 1.67 | |
| Average | | 1.53 | |

EVA with HMI
240 minutes

| Sample | Film (g) | Wet (g) | Absorption (g/g) |
|---|---|---|---|
| 1 | 0.20 | 18.84 | 85.25 |
| 2 | 0.20 | 19.00 | 86.45 |
| 3 | 0.20 | 17.82 | 80.19 |
| | | | Avg Abs (g/g) |
| | | | 83.96 |
| Blank 1 | 0.24 | 1.25 | |
| Blank 2 | 0.24 | 1.42 | |
| Average | | 1.33 | |

EVA with LMI
240 minutes

| Sample | Film (g) | Wet (g) | Absorption (g/g) |
|---|---|---|---|
| 1 | 0.20 | 6.70 | 25.30 |
| 2 | 0.20 | 6.39 | 24.43 |
| 3 | 0.20 | 6.35 | 24.22 |
| | | | Avg Abs (g/g) |
| | | | 24.65 |
| Blank 1 | 0.24 | 1.25 | |
| Blank 2 | 0.24 | 1.42 | |
| Average | | 1.33 | |

EVA with HMI
480 minutes

| Sample | Film (g) | Wet (g) | Absorption (g/g) |
|---|---|---|---|
| 1 | 0.20 | 19.31 | 88.56 |
| 2 | 0.20 | 19.34 | 87.80 |
| 3 | 0.20 | 19.47 | 88.92 |
| | | | Avg Abs (g/g) |
| | | | 88.42 |
| Blank 1 | 0.23 | 1.57 | |
| Blank 2 | 0.24 | 1.41 | |
| Average | | 1.49 | |

EVA with LMI
480 minutes

| Sample | Film (g) | Wet (g) | Absorption (g/g) |
|---|---|---|---|
| 1 | 0.20 | 8.11 | 31.75 |
| 2 | 0.20 | 7.64 | 29.76 |
| 3 | 0.20 | 6.68 | 25.32 |
| | | | Avg Abs (g/g) |
| | | | 28.94 |
| Blank 1 | 0.23 | 1.57 | |
| Blank 2 | 0.24 | 1.41 | |
| Average | | 1.49 | |

EVA with HMI
1380 minutes

| Sample | Film (g) | Wet (g) | Absorption (g/g) |
|---|---|---|---|
| 1 | 0.20 | 17.85 | 82.79 |
| 2 | 0.20 | 20.55 | 94.61 |
| 3 | 0.20 | 20.77 | 95.72 |
| | | | Avg Abs (g/g) |
| | | | 91.04 |
| Blank 1 | 0.26 | 1.41 | |
| Blank 2 | 0.23 | 1.44 | |
| Average | | 1.43 | |

EVA with LMI
1380 minutes

| Sample | Film (g) | Wet (g) | Absorption (g/g) |
|---|---|---|---|
| 1 | 0.20 | 7.19 | 27.65 |
| 2 | 0.20 | 7.14 | 27.47 |
| 3 | 0.20 | 6.83 | 26.35 |
| | | | Avg Abs (g/g) |
| | | | 27.16 |
| Blank 1 | 0.26 | 1.41 | |
| Blank 2 | 0.23 | 1.44 | |
| Average | | 1.43 | |

EVA with HMI
2820 minutes

| Sample | Film (g) | Wet (g) | Absorption (g/g) |
|---|---|---|---|
| 1 | 0.20 | 20.74 | 96.26 |
| 2 | 0.20 | 20.86 | 94.46 |
| 3 | 0.20 | 21.11 | 98.03 |
| | | | Avg Abs (g/g) |
| | | | 96.27 |
| Blank 1 | 0.24 | 1.52 | |
| Blank 2 | 0.23 | 1.25 | |
| Average | | 1.39 | |

EVA with LMI
2820 minutes

| Sample | Film (g) | Wet (g) | Absorption (g/g) |
|---|---|---|---|
| 1 | 0.20 | 6.44 | 24.28 |
| 2 | 0.20 | 6.55 | 24.29 |
| 3 | 0.20 | 6.58 | 24.98 |
| | | | Avg Abs (g/g) |
| | | | 24.52 |
| Blank 1 | 0.24 | 1.52 | |
| Blank 2 | 0.23 | 1.25 | |
| Average | | 1.39 | |

TABLE B
(20 mil thickness films)
EVA with HMI
5 minutes

| Sample | Film (g) | Wet (g) | Absorption (g/g) |
|---|---|---|---|
| 1 | 0.20 | 4.78 | 18.92 |
| 2 | 0.20 | 5.14 | 20.74 |
| 3 | 0.20 | 4.38 | 16.74 |
| | | | Avg Abs (g/g) |
| | | | 18.80 |
| Blank 1 | 0.26 | 0.85 | |
| Blank 2 | 0.25 | 0.78 | |
| Average | | 0.81 | |

EVA with LMI
5 minutes

| Sample | Film (g) | Wet (g) | Absorption (g/g) |
|---|---|---|---|
| 1 | 0.20 | 4.49 | 17.49 |
| 2 | 0.20 | 4.47 | 17.37 |
| 3 | 0.20 | 4.31 | 16.07 |
| | | | Avg Abs (g/g) |
| | | | 16.98 |
| Blank 1 | 0.26 | 0.85 | |
| Blank 2 | 0.25 | 0.78 | |
| Average | | 0.81 | |

EVA with HMI
10 minutes

| Sample | Film (g) | Wet (g) | Absorption (g/g) |
|---|---|---|---|
| 1 | 0.20 | 5.94 | 23.42 |
| 2 | 0.20 | 5.95 | 23.49 |
| 3 | 0.20 | 6.06 | 23.89 |
| | | | Avg Abs (g/g) |
| | | | 23.60 |
| Blank 1 | 0.25 | 1.08 | |
| Blank 2 | 0.25 | 0.99 | |
| Average | | 1.03 | |

EVA with LMI
10 minutes

| Sample | Film (g) | Wet (g) | Absorption (g/g) |
|---|---|---|---|
| 1 | 0.20 | 6.10 | 24.73 |
| 2 | 0.21 | 6.41 | 25.26 |
| 3 | 0.20 | 6.79 | 27.36 |
| | | | Avg Abs (g/g) |
| | | | 25.78 |
| Blank 1 | 0.25 | 1.08 | |
| Blank 2 | 0.25 | 0.99 | |
| Average | | 1.03 | |

EVA with HMI
15 minutes

| Sample | Film (g) | Wet (g) | Absorption (g/g) |
|---|---|---|---|
| 1 | 0.20 | 12.36 | 53.38 |
| 2 | 0.20 | 12.18 | 51.94 |
| 3 | 0.20 | 11.48 | 50.02 |
| | | | Avg Abs (g/g) |
| | | | 51.78 |
| Blank 1 | 0.23 | 1.43 | |
| Blank 2 | 0.23 | 1.43 | |
| Average | | 1.43 | |

EVA with LMI
15 minutes

| Sample | Film (g) | Wet (g) | Absorption (g/g) |
|---|---|---|---|
| 1 | 0.20 | 7.758 | 31.12 |
| 2 | 0.20 | 9.554 | 40.03 |
| 3 | 0.20 | 10.53 | 44.49 |
| | | | Avg Abs (g/g) |
| | | | 38.55 |
| Blank 1 | 0.23 | 1.43 | |
| Blank 2 | 0.23 | 1.43 | |
| Average | | 1.43 | |

EVA with HMI
20 minutes

| Sample | Film (g) | Wet (g) | Absorption (g/g) |
|---|---|---|---|
| 1 | 0.20 | 9.00 | 39.48 |
| 2 | 0.20 | 8.77 | 36.78 |
| 3 | 0.20 | 9.25 | 40.58 |
| | | | Avg Abs (g/g) |
| | | | 38.94 |
| Blank 1 | 0.25 | 1.13 | |
| Blank 2 | 0.25 | 1.00 | |
| Average | | 1.06 | |

EVA with LMI
20 minutes

| Sample | Film (g) | Wet (g) | Absorption (g/g) |
|---|---|---|---|
| 1 | 0.20 | 7.51 | 30.91 |
| 2 | 0.20 | 7.94 | 32.86 |
| 3 | 0.20 | 7.49 | 31.15 |
| | | | Avg Abs (g/g) |
| | | | 31.64 |
| Blank 1 | 0.25 | 1.13 | |
| Blank 2 | 0.25 | 1.00 | |
| Average | | 1.06 | |

EVA with HMI
25 minutes

| Sample | Film (g) | Wet (g) | Absorption (g/g) |
|---|---|---|---|
| 1 | 0.20 | 10.12 | 43.45 |
| 2 | 0.20 | 9.17 | 38.30 |
| 3 | 0.20 | 9.83 | 40.02 |
| | | | Avg Abs (g/g) |
| | | | 41.25 |
| Blank 1 | 0.25 | 1.16 | |
| Blank 2 | 0.25 | 1.22 | |
| Average | | 1.19 | |

EVA with LMI
25 minutes

| Sample | Film (g) | Wet (g) | Absorption (g/g) |
|---|---|---|---|
| 1 | 0.20 | 9.59 | 41.65 |
| 2 | 0.20 | 10.45 | 46.24 |
| 3 | 0.20 | 9.91 | 42.19 |
| | | | Avg Abs (g/g) |
| | | | 43.36 |
| Blank 1 | 0.25 | 1.16 | |
| Blank 2 | 0.25 | 1.22 | |
| Average | | 1.19 | |

EVA with HMI
30 minutes

| Sample | Film (g) | Wet (g) | Absorption (g/g) |
|---|---|---|---|
| 1 | 0.20 | 10.24 | 43.41 |
| 2 | 0.20 | 10.27 | 44.03 |
| 3 | 0.20 | 10.49 | 45.12 |
| | | | Avg Abs (g/g) |
| | | | 44.19 |
| Blank 1 | 0.25 | 1.38 | |
| Blank 2 | 0.25 | 1.06 | |
| Average | | 1.22 | |

EVA with LMI
30 minutes

| Sample | Film (g) | Wet (g) | Absorption (g/g) |
|---|---|---|---|
| 1 | 0.20 | 10.31 | 43.77 |
| 2 | 0.20 | 10.78 | 47.26 |
| 3 | 0.20 | 10.63 | 46.05 |
| | | | Avg Abs (g/g) |
| | | | 45.69 |
| Blank 1 | 0.25 | 1.38 | |
| Blank 2 | 0.25 | 1.06 | |
| Average | | 1.22 | |

EVA with HMI
60 minutes

| Sample | Film (g) | Wet (g) | Absorption (g/g) |
|---|---|---|---|
| 1 | 0.20 | 14.62 | 66.06 |
| 2 | 0.20 | 15.11 | 68.16 |
| 3 | 0.20 | 15.14 | 67.29 |
| | | | Avg Abs (g/g) |
| | | | 67.17 |
| Blank 1 | 0.24 | 1.35 | |
| Blank 2 | 0.24 | 1.21 | |
| Average | | 1.28 | |

EVA with LMI
60 minutes

| Sample | Film (g) | Wet (g) | Absorption (g/g) |
|---|---|---|---|
| 1 | 0.20 | 12.54 | 54.75 |
| 2 | 0.20 | 12.27 | 53.41 |
| 3 | 0.20 | 11.22 | 49.49 |
| | | | Avg Abs (g/g) |
| | | | 52.55 |
| Blank 1 | 0.24 | 1.35 | |
| Blank 2 | 0.24 | 1.21 | |
| Average | | 1.28 | |

EVA with HMI
120 minutes

| Sample | Film (g) | Wet (g) | Absorption (g/g) |
|---|---|---|---|
| 1 | 0.20 | 15.77 | 69.83 |
| 2 | 0.20 | 15.83 | 69.78 |
| 3 | 0.20 | 15.28 | 68.80 |
| | | | Avg Abs (g/g) |
| | | | 69.47 |
| Blank 1 | 0.24 | 1.40 | |
| Blank 2 | 0.24 | 1.67 | |
| Average | | 1.53 | |

EVA with LMI
120 minutes

| Sample | Film (g) | Wet (g) | Absorption (g/g) |
|---|---|---|---|
| 1 | 0.20 | 14.15 | 61.47 |
| 2 | 0.20 | 13.31 | 57.88 |
| 3 | 0.20 | 13.90 | 60.81 |
| | | | Avg Abs (g/g) |
| | | | 60.05 |
| Blank 1 | 0.24 | 1.40 | |
| Blank 2 | 0.24 | 1.67 | |
| Average | | 1.53 | |

EVA with HMI
240 minutes

| Sample | Film (g) | Wet (g) | Absorption (g/g) |
|---|---|---|---|
| 1 | 0.20 | 16.80 | 75.19 |
| 2 | 0.20 | 17.21 | 77.22 |
| 3 | 0.20 | 16.45 | 74.59 |
| | | | Avg Abs (g/g) |
| | | | 75.67 |
| Blank 1 | 0.24 | 1.25 | |
| Blank 2 | 0.24 | 1.42 | |
| Average | | 1.33 | |

EVA with LMI
240 minutes

| Sample | Film (g) | Wet (g) | Absorption (g/g) |
|---|---|---|---|
| 1 | 0.20 | 14.22 | 62.17 |
| 2 | 0.20 | 14.87 | 66.33 |
| 3 | 0.20 | 14.88 | 66.04 |
| | | | Avg Abs (g/g) |
| | | | 64.85 |
| Blank 1 | 0.24 | 1.25 | |
| Blank 2 | 0.24 | 1.42 | |
| Average | | 1.33 | |

EVA with HMI
480 minutes

| Sample | Film (g) | Wet (g) | Absorption (g/g) |
|---|---|---|---|
| 1 | 0.20 | 18.17 | 83.66 |
| 2 | 0.20 | 18.49 | 84.84 |
| 3 | 0.20 | 17.73 | 79.37 |
| | | | Avg Abs (g/g) |
| | | | 82.63 |
| Blank 1 | 0.23 | 1.57 | |
| Blank 2 | 0.24 | 1.41 | |
| Average | | 1.49 | |

EVA with LMI
480 minutes

| Sample | Film (g) | Wet (g) | Absorption (g/g) |
|---|---|---|---|
| 1 | 0.20 | 16.02 | 72.03 |
| 2 | 0.20 | 16.47 | 73.54 |
| 3 | 0.20 | 15.49 | 69.69 |
| | | | Avg Abs (g/g) |
| | | | 71.75 |
| Blank 1 | 0.23 | 1.57 | |
| Blank 2 | 0.24 | 1.41 | |
| Average | | 1.49 | |

EVA with HMI
1380 minutes

| Sample | Film (g) | Wet (g) | Absorption (g/g) |
|---|---|---|---|
| 1 | 0.20 | 19.92 | 91.94 |
| 2 | 0.20 | 19.82 | 89.15 |
| 3 | 0.20 | 19.59 | 90.25 |
| | | | Avg Abs (g/g) |
| | | | 90.45 |
| Blank 1 | 0.26 | 1.41 | |
| Blank 2 | 0.23 | 1.44 | |
| Average | | 1.43 | |

EVA with LMI
1380 minutes

| Sample | Film (g) | Wet (g) | Absorption (g/g) |
|---|---|---|---|
| 1 | 0.20 | 16.11 | 73.19 |
| 2 | 0.20 | 16.24 | 73.46 |
| 3 | 0.20 | 15.77 | 70.01 |
| | | | Avg Abs (g/g) |
| | | | 72.22 |
| Blank 1 | 0.26 | 1.41 | |
| Blank 2 | 0.23 | 1.44 | |
| Average | | 1.43 | |

EVA with HMI
2820 minutes

| Sample | Film (g) | Wet (g) | Absorption (g/g) |
|---|---|---|---|
| 1 | 0.20 | 20.39 | 92.62 |
| 2 | 0.20 | 19.60 | 90.52 |
| 3 | 0.20 | 19.34 | 89.05 |
| | | | Avg Abs (g/g) |
| | | | 90.73 |
| Blank 1 | 0.24 | 1.52 | |
| Blank 2 | 0.23 | 1.25 | |
| Average | | 1.39 | |

EVA with LMI
2820 minutes

| Sample | Film (g) | Wet (g) | Absorption (g/g) |
|---|---|---|---|
| 1 | 0.20 | 16.20 | 73.83 |
| 2 | 0.20 | 16.33 | 73.73 |
| 3 | 0.20 | 16.04 | 72.25 |
| | | | Avg Abs (g/g) |
| | | | 73.27 |
| Blank 1 | 0.24 | 1.52 | |
| Blank 2 | 0.23 | 1.25 | |
| Average | | 1.39 | |

As can be seen from Tables A and B, for any specific absorbency time, when 2 films of EVA+SAP are equal but for one had an EVA with a HMI and the other had an EVA with a LMI, then a far superior average absorption for the short term, i.e., 15 minutes of less, and a far superior average absorption for the long term, i.e., 60 minutes or longer, was exhibited for the 20 mil films that had the EVA with the HMI as compared to the 20 mil films that had the EVA with the LMI. For the 10 mil films, a far superior average absorption for all times tested was always exhibited for films that had the EVA with the HMI as compared to films that had the EVA with the LMI. This is summarized in Table C below.

TABLE C
(Summary of Avg Abs from Tables A and B)

| Time | Avg Abs (g/g) | |
|---|---|---|
| (min) | 10 mil Films | 20 mil Films |

| TB test | HMI EVA | LMI EVA | HMI EVA | LMI EVA |
|---|---|---|---|---|
| 5 | 33.32 | 10.32 | 18.80 | 16.98 |
| 10 | 45.66 | 12.13 | 23.60 | 25.78 |
| 15 | 67.34 | 15.01 | 51.78 | 38.55 |
| 20 | 56.37 | 15.48 | 38.94 | 31.64 |
| 25 | 58.65 | 15.13 | 41.25* | 43.36 |
| 30 | 59.11 | 18.25 | 44.19* | 45.69 |
| 60 | 76.12 | 19.22 | 67.17 | 52.55 |
| 120 | 75.25 | 21.33 | 69.47 | 60.05 |
| 240 | 83.96 | 24.65 | 75.67 | 64.85 |
| 480 | 88.42 | 28.94 | 82.63 | 71.75 |
| 1380 | 91.04 | 27.16 | 90.45 | 72.22 |
| 2820 | 96.27 | 24.52 | 90.73 | 73.27 |

*It appears a gel blocking effect occurred at 25 and 30 minutes for the thicker 20 mil films with an EVA having a HMI, as at these times, the 20 mil films that had an EVA with an HMI did not exhibit a superior average absorption to the 20 mil films that had an EVA with a LMI.

It will therefore be readily understood by those persons skilled in the art that the present invention is susceptible of broad utility and application. Many embodiments and adaptations of the present invention other than those herein described, as well as many variations, modifications and equivalent arrangements will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being defined by the claims appended hereto and the equivalents thereof.

What is claimed is:

1. An absorbent material, comprising at least about 30 weight percent of superabsorbent polymer, thermoplastic polymer binder resin, and about 0.1 to about 10 weight percent water, wherein the absorbent material absorbs deionized water to at least about 70 percent of capacity within about 20 minutes after exposure to the deionized water.

2. The absorbent material of claim 1, wherein the superabsorbent polymer comprises at least one monomer selected from the group consisting of acrylonitrile groups, anhydride groups, carboxylic acid groups, sulfonic acid groups, and combinations thereof.

3. The absorbent material of claim 2, wherein the carboxylic acid groups are selected from the group consisting of methacrylic acids, salts of methacrylic acids, acrylic acids, salts of acrylic acids, and combinations thereof.

4. The absorbent material of claim 3, wherein the superabsorbent polymer is sodium polyacrylate.

5. The absorbent material of claim 1, wherein the binder resin is a polymer of at least one monomer selected from the group consisting of hydrophobic monomers.

6. The absorbent material of claim 5, wherein the binder resin is a polyolefin.

7. The absorbent material of claim 6, wherein the polyolefin is selected from the group consisting of ethylene vinyl acetate copolymer, ethylene methyl acrylate copolymer, ethylene butyl acrylate copolymer, ethylene ethyl acrylate copolymer, polypropylene, very low density linear polyethylene, linear low density polyethylene, low density polyethylene, medium density polyethylene, high density polyethylene, and combinations thereof.

8. The absorbent material of claim 7, wherein the ethylene vinyl acetate copolymer has a melt index greater than about 300.

9. The absorbent material of claim 1, wherein the superabsorbent polymer has a particle size up to about 1000 μm.

10. The absorbent material of claim 9, wherein the superabsorbent polymer has a particle size up to about 800 μm.

11. The absorbent material of claim 1, wherein the superabsorbent polymer has a particle size of about 100 μm or less.

12. The absorbent material of claim 11, wherein the superabsorbent polymer has a particle size of about 50 μm or less.

13. The absorbent material of claim 1, wherein a minor amount of an inorganic particulate is incorporated into the absorbent material to minimize agglomeration.

14. The absorbent material of claim 13, wherein the inorganic particulate is silica.

15. The absorbent material of claim 1, wherein the absorbent material has a form selected from the group consisting of films, strands, and pellets.

16. The absorbent material of claim 15, wherein the absorbent material is a film selected from the group consisting of heat-shrinkable film, non-heat-shrinkable film, and combinations thereof.

17. The absorbent material of claim 1, wherein the absorbent material comprises up to about 90 weight percent of the superabsorbent polymer.

18. The absorbent material of claim 1, wherein the absorbent material is free of polyesters and of rubber polymers.

19. An article of manufacture comprising an absorbent material, having at least about 30 weight percent of superabsorbent polymer, thermoplastic polymer binder resin, and about 0.1 to about 10 weight percent water, wherein the absorbent material absorbs deionized water to at least about 70 percent of capacity within about 20 minutes after exposure to the deionized water.

20. The article of manufacture of claim 19, wherein the article is selected from the group consisting of a sanitary article, a sealing composite article, a water blocking tape article, an agricultural article, a filtration sheet article, an absorbent liner article, a packaging material article for packaging items requiring moisture, a filler material article, and a coating material article.

21. The sanitary article of claim 20, employed in throwaway baby diapers, incontinence garments, bed pads, sanitary napkins, bandages, wound dressings, surgical drapes, or clean-up pads.

22. The sealing composite article of claim 20, employed in wine corks, boats, houses, plumbing, water stops, caulking, gaskets, hydraulic cement, gutters, flat tire repair, or water proofing composites.

23. The water blocking tape article of claim 20, employed in fiber optic cables or power transmission cables.

24. The agricultural article of claim 20, employed in a controlled release carrier for insecticides, herbicides, or pesticides, or in a soil amendment in agricultural fields to improve the capability of soils to keep water and nutrients near or with the roots of plants.

25. The filtration sheet article of claim 20, employed in removal of water or moisture from gasoline, fuel, oil, or organic solvent.

26. The absorbent liner article of claim 20, employed in food packaging, astroturf, dikes, erosion control, irrigation systems, book repair, hydraulic devices, roofs, gas tanks, slaughter houses, mildew protectors, or shipping containers.

27. The packaging material article of claim 20, employed in packaging cut flowers.

28. The filler material article of claim 20, employed in recording materials, batteries, brush fibers, fire place logs, furniture, gel transformers, tractor tires, diet pills, cast reinforcement, ballast, capillary devices, humidity control devices, or indoor/outdoor carpets.

29. The coating material article of claim 20, employed in antifouling coatings, paint additives, or thickeners.

30. A method for making an absorbent material comprising the steps of:
(a) combining a thermoplastic polymer binder resin, a superabsorbent polymer, and water in a twin screw extrusion mechanism into a blended composition;
(b) extruding the composition through extrusion openings in the twin screw extrusion mechanism; and
(c) forming an absorbent material which absorbs deionized water to at least about 70 percent of capacity within about 20 minutes after exposure to the deionized water.

31. The method of claim 30, further comprising after step (b) rapidly cooling the extruded resultant with non-liquid quenching means to form the absorbent material.

32. The method of claim 30, further comprising the step of vacuum venting to remove excess moisture in the extrusion mechanism to maintain a moisture level in the absorbent material from about 0.1 to about 10 weight percent water.

33. The method of claim 30, wherein the step of combining the binder resin and the superabsorbent polymer comprises that the binder resin is a polyolefin binder resin being combined with at least about 30 weight percent of the superabsorbent polymer.

34. The method of claim 33, wherein the polyolefin binder resin is combined with up to about 90 weight percent of the superabsorbent polymer.

35. The method of claim 30, wherein the step of extruding comprises extruding through extrusion openings of predetermined shape such that the absorbent material has a form selected from the group consisting of films and strands.

36. The method of claim 35, further comprising the step of cutting the strands into pellets.

37. The method of claim 35, wherein the film has sufficient tensile strength to maintain self-supporting continuity in the film.

38. The method of claim 30, further comprising a processing step so that the absorbent material is a film selected from the group consisting of heat-shrinkable film, non-heat-shrinkable film, and combinations thereof.

39. The method of claim 30, wherein the superabsorbent polymer comprises at least one monomer selected from the group consisting of acrylonitrile groups, anhydride groups, carboxylic acid groups, sulfonic acid groups, and combinations thereof.

40. The absorbent material of claim 39, wherein the carboxylic acid groups are selected from the group consisting of methacrylic acids, salts of methacrylic acids, acrylic acids, salts of acrylic acids, and combinations thereof.

41. The method of claim 40, wherein the superabsorbent polymer is sodium polyacrylate.

42. The method of claim 30, wherein the binder resin is a polymer of at least one monomer selected from the group consisting of hydrophobic monomers.

43. The method of claim 42, wherein the binder resin is a polyolefin.

44. The method of claim 43, wherein the polyolefin is selected from the group consisting of ethylene vinyl acetate copolymer, ethylene methyl acrylate copolymer, ethylene butyl acrylate copolymer, ethylene ethyl acrylate copolymer, polypropylene, very low density linear polyethylene, linear low density polyethylene, low density polyethylene, medium density polyethylene, high density polyethylene, and combinations thereof.

45. The method of claim 44, wherein the ethylene vinyl acetate copolymer has a melt index greater than about 300.

46. The method of claim 30, wherein the superabsorbent polymer has a particle size up to about 1000 μm.

47. The method of claim 46, wherein the superabsorbent polymer has a particle size up to about 800 μm.

48. The method of claim 30, wherein the superabsorbent polymer has a particle size of about 100 μm or less.

49. The method of claim 48, wherein the superabsorbent polymer has a particle size of about 50 μm or less.

50. The method of claim 30, wherein a minor amount of an inorganic particulate is incorporated into the absorbent material to minimize agglomeration.

51. The method of claim 50, wherein the inorganic particulate is silica.

52. The method of claim 30, wherein the absorbent material is free of polyesters and of rubber polymers.

53. The method of claim 30, wherein the absorbent material is formed into an article selected from the group consisting of a sanitary article, a sealing composite article, a water blocking tape article, an agricultural article, a filtration sheet article, an absorbent liner article, a packaging material article for packaging items requiring moisture, a filler material article, and a coating material article.

54. The method of claim 53, wherein the sanitary article is employed in throwaway baby diapers, incontinence garments, bed pads, sanitary napkins, bandages, wound dressings, surgical drapes, or clean-up pads.

55. The method of claim 53, wherein the sealing composite article is employed in wine corks, boats, houses, plumbing, water stops, caulking, gaskets, hydraulic cement, gutters, flat tire repair, or water proofing composites.

56. The method of claim 53, wherein the water blocking tape article is employed in fiber optic cables or power transmission cables.

57. The method of claim 53, wherein the agricultural article is employed in a controlled release carrier for insecticides, herbicides, or pesticides, or in a soil amendment in agricultural fields to improve the capability of soils to keep water and nutrients near or with the roots of plants.

58. The method of claim 53, wherein the filtration sheet article is employed in removal of water or moisture from gasoline, fuel, oil, or organic solvent.

59. The method of claim 53, wherein the absorbent liner article is employed in food packaging, astroturf, dikes, erosion control, irrigation systems, book repair, hydraulic devices, roofs, gas tanks, slaughter houses, mildew protectors, or shipping containers.

60. The method of claim 53, wherein the packaging material article is employed in packaging cut flowers.

61. The method of claim 53, wherein the filler material article is employed in recording materials, batteries, brush fibers, fire place logs, furniture, gel transformers, tractor tires, diet pills, cast reinforcement, ballast, capillary devices, humidity control devices, or indoor/outdoor carpets.

62. The method of claim 53, wherein the coating material article is employed in antifouling coatings, paint additives, or thickeners.

63. A method of providing for the prevention of the migration of liquid within a cable having at least one component, comprising the steps of:
(a) extruding a composition comprising from about 30 weight percent to about 90 weight percent of a superabsorbent polymer and a polyolefin binder resin, by a twin-screw extrusion mechanism;
(b) rapidly cooling the extruded resultant with non-liquid quenching means to form an absorbent material that absorbs deionized water to at least about 70 percent of capacity within about 20 minutes after exposure to the deionized water;
(c) providing at least one cable component with the absorbent material,
whereby the absorbent material absorbs liquid within said cable and provides liquid-blocking properties upon liquid absorption to prevent the migration of liquid through said cable.

64. The method of claim 63, wherein step (a) further comprises vacuum venting to remove excess moisture in the extrusion process to maintain the moisture level at from about 0.1 to about 10 weight percent water in the absorbent material.

65. The method of claim 63, wherein step (c) further comprises enveloping the component with the absorbent material.

66. The method of claim 63, wherein step (c) further comprises placing a tape of the absorbent material film along the component.

67. The method of claim 63, wherein the absorbent material is a film that has sufficient tensile strength to maintain self-supporting continuity without holes developing in the absorbent material film.

68. The method of claim 63, further comprising a processing step so that the absorbent material is a film selected from the group of consisting heat-shrinkable film, non-heat-shrinkable film, and combinations thereof.

69. The method of claim 63, wherein the absorbent material is free of polyesters and of rubber polymers.

70. The method of claim 63, wherein the superabsorbent polymer has a particle size up to about 1000 μm.

71. The method of claim 70, wherein the superabsorbent polymer has a particle size up to about 800 μm.

72. The method of claim 63, wherein the superabsorbent polymer has a particle size of about 100 μm or less.

73. The method of claim 72, wherein the superabsorbent polymer has a particle size of about 50 μm or less.
